(19) United States
(12) Patent Application Publication
(10) Pub. No.: US 2004/0038890 A1
(43) Pub. Date: Feb. 26, 2004

(54) Human voltage-gated potassium channel subunit

(76) Inventors: Jayashree Aiyar, Wilmington, DE (US); Jiesheng Kang, Piscataway, NJ (US)

Correspondence Address: ASTRA ZENECA PHARMACEUTICALS LP, GLOBAL INTELLECTUAL PROPERTY, 1800 CONCORD PIKE, WILMINGTON, DE, 19850-5437, US

(21) Appl. No.: 10/435,935

(22) Filed: May 12, 2003

Related U.S. Application Data
(63) Continuation of application No. 09/074,878, filed on May 8, 1998, now abandoned.

(51) Int. Cl. A61K 38/17; C07K 14/705; C12P 21/02; C12N 5/06; C07H 21/04

(52) U.S. Cl. 514/12; 435/69.1; 435/320.1; 435/325; 530/350; 536/23.5

(57) ABSTRACT

A novel human voltage-gated potassium channel subunit is provided. A full length cDNA which encodes the novel potassium channel polypeptide is disclosed as well as the interior structural region and the amino acid residue sequence of the voltage-gated potassium channel biomolecule. Methods are provided to identify compounds that modulate the pharmacological activity of the potassium channel subunit and hence regulate potassium channel biological activity.

### CROSS-REFERENCE TO RELATED APPLICATIONS

This application is a continuation of application Ser. No. 09/074,878, filed May 8, 1998, which is incorporated herein by reference in its entirety.

### FIELD OF THE INVENTION

The present invention relates to nucleic acid and amino acid sequences of a novel human voltage-gated potassium channel polypeptide and to the use of these sequences to identify compounds that modulate the pharmacological activity of the native biomolecule as well as the biological activity of a potassium channel of which hKv9.3 is an integral member. The invention is also related to the diagnosis, study, prevention, and treatment of pathophysiological disorders related to or mediated by potassium channels.

### BACKGROUND OF THE INVENTION

Potassium channels are integral membrane proteins of great molecular and functional diversity and are present in virtually all mammalian cells. Depending on the sub-family to which a given potassium channel belongs, it can be activated by a change in the membrane potential, an increase in the intracellular concentration of Ca²⁺, or binding of ligands to their receptors including acetycholine, adrenaline, dopamine, galanin, calcitonin gene-related peptide, somatostatin, and ATP. The pharmacological identity of various voltage-gated potassium-channels is established by their sensitivity to standard compounds capable of blocking one or more types of potassium channels. These compounds, known as potassium-channel blockers, include tetraethylammonium chloride (TEA), 4-aminopyridine (4-AP, as well as 2-AP and 3-AP), 3,4- and 2,3-diaminopyridine, BaCl₂, CsCl, strychnine, phencyclidine, pyridostigmine, 9-aminoacridine, DuP-996 (3,3-bis (4-pyridinylmethyl)-1-phenylindolin-2-one; linopiridine), clofilium, quinidine, aminoquinolines and quinine. Extensive molecular and functional diversity renders potassium channels a potential target of drug discovery for a large variety of pathophysiological indications. Toral, J., et al., Use of Cultured Human Neuroblastoma Cells in Rapid Discovery of the Voltage-gated Potassium-channel Blockers, J. Pharm. Pharmacol., 46:731 (1994).

There is a great diversity of voltage-gated K⁺ channels in muscular cells which have different biophysical, regulational and pharmacological properties. Potassium channels are generally assembled as tetramers of subunits. The concept that potassium channel subunits associate only with other closely related sub-family members has been set forth and described. Xu, J., et al., J. Biol. Chem., 270(42):24761 (1995). Kv1 subfamily members, for example, have previously been accepted to interact best with other Kv1-subunits, and less efficiently with Kv2, Kv3 or Kv4 subunits. However, a factor of functional diversity can be achieved by the formation of heteromultimeric channels with properties distinct from those of their parent homomultimers. Hugnot, J. P., et al., Kv8.1 a New Neuronal Potassium Channel Subunit with Specific Inhibitory Properties Towards Shab and Shaw Channels, EMBO, 15(13):3322 (1996). Members of the more recently described sub-families namely, Kv5, Kv6, Kv7, Kv8 and Kv9, have been shown to heteromultimerize with Kv2 and Kv3 sub-family members. These resulting potassium channels have been shown to have novel functional properties. Patel, A. J., et al., EMBO, 16(22):6615 (1997); Salinas, M., et al., J. Biol. Chem., 272(39): 24371 (1997); Salinas, M., et al., J. Biol. Chem., 272(13): 8774 (1997).

Oxygen is an essential requirement for cell survival. However, Organisms have the ability to rapidly adapt to hypoxia. Potassium channels play a key role in adaptive hypoxic mechanisms and are widely believed to be involved in the sensing of oxygen. One such adaptation is the hypoxia-induced vasoconstriction of resistance pulmonary artery (PA) smooth muscle which leads to a redistribution of the non-oxygenated blood towards better ventilated regions of the lung. In the fetus, for example, hypoxic pulmonary vasoconstriction (HPV) diverts blood through the ductus arteriosus and is essential for fetal survival. Although HPV fulfills an essential physiological function, it also contributes to the development of pulmonary hypertension in patients with chronic obstructive lung diseases (e.g., chronic bronchitis, emphysema) and people living at high altitudes. It has been demonstrated that hypoxic vasoconstriction of resistance PA smooth muscle cells is mainly mediated by the closing of voltage-dependent potassium channels leading to cell depolarization, calcium influx and myocyte contraction. Chronic hypoxia has also been associated with reduced delayed-rectifier K⁺ current in rat pulmonary artery smooth muscle cells. Moreover, pulmonary hypertension is a dramatic disease which ultimately provokes right heart failure and death within 2 to 5 years. It occurs in people living at high altitude and in patients suffering from chronic obstructive lung diseases such as chronic bronchitis and emphysema. Furthermore, it has been demonstrated that anorexic agents such as aminorex fumarate and DFF have caused an epidemic of pulmonary hypertension in Europe. Interestingly, these compounds have been recently shown to inhibit K⁺ currents in rat pulmonary artery myocytes and to cause pulmonary vasoconstriction. Patel, A. J., et al., EMBO,16(22):6615 (1997).

Patel, et al., have reported the molecular cloning, characterization and regulation by hypoxia of a voltage- and ATP-dependent Shab-related potassium channel subunit, rKv9.3, isolated from rat PA smooth muscle cells. The molecular identification of Kv9.3 is reported to be of tremendous importance in the understanding of pulmonary hypertension and in the design of novel therapeutic strategies. Patel, A. J., et al., EMBO,16(22):6615 (1997). However, the reported modulatory subunit, rKv5.1, is a rat isolate. The availability of a functional human homolog will be ideal for such drug screening as well as diagnosis, study, prevention, and treatment of pathophysiological disorders related to or mediated by the biological molecule. The availability of a functional assay with a considerable through-put is an indispensable part of any drug discovery program focusing on potassium channel modulators. Such an assay should be able to screen hundreds of compounds on a routine basis. Toral, J., et al., Use of Cultured Human Neuroblastoma Cells in Rapid Discovery of the Voltage-gated Potassium-channel Blockers, J. Pharm. Pharmacol., 46:731 (1994).

### SUMMARY OF THE INVENTION

The present invention is directed to an isolated and purified polynucleotide molecule, which encodes a polypeptide of a potassium channel, or a biologically-effective fragment thereof comprising a nucleic acid sequence encoding the polypeptide having the sequence substantially as depicted in SEQ ID NO:3 or a pharmacologically acive fragment thereof. Isolated and purified polynucleotides of the present invention include but are not limited to SEQ ID NO: 1 (hKv9.3—human voltage-gated potassium channel polypeptide cDNA) and SEQ ID NO:2 (hKv9.3—human voltage-gated potassium channel polypeptide structural coding region).

In addition, the current invention is directed to a purified polypeptide comprising the amino acid sequence substantially as depicted in SEQ ID NO:3.

The invention is further directed to a host cell containing an expression vector for expression of a potassium channel polypeptide, wherein said vector contains a polynucleotide comprising a nucleic acid sequence encoding the polypeptide of a potassium channel having the sequence substantially as depicted in SEQ ID NO:3 or a pharmacologically active fragment thereof. The invention is also directed to a method for producing a potassium channel polypeptide having the amino acid sequence substantially as depicted in SEQ ID NO:3 by culturing said host cell under conditions suitable for the expression of said polypeptide, and recovering said polypeptide from the host cell culture.

The instant invention is further directed to a method of identifying pharmacological activity contributed by hKv9.3 to a potassium channel, comprising:

a) providing a first host cell which expresses at least one potassium channel subunit to produce a first potassium channel; and

b) providing a second host cell which co-expresses a potassium channel polypeptide comprising the sequence substantially as depicted in SEQ ID NO:3 along with the said at least one potassium channel subunit from step a) to produce a second potassium channel; and

c) comparing the biological activity of the first potassium channel to the biological activity of the second potassium channel and identifying the pharmacological activity contributed by hKv9.3 to the potassium channel.

Further, the invention is further directed to a method of identifying compounds that modulate the pharmacological activity of hKv9.3, comprising:

a) providing a first host cell which expresses at least one potassium channel subunit to produce a first potassium channel; and

b) providing a second host cell which co-expresses a potassium channel polypeptide comprising the sequence substantially as depicted in SEQ ID NO:3 with the said at least one potassium channel subunit to produce a second potassium channel; and

c) comparing the biological activity of the first potassium channel to the biological activity of the second potassium channel and identifying the pharmacological activity contributed by hKv9.3 to the potassium channel; and

d) combining a candidate compound modulator of hKv9.3 pharmacological activity with said second host cell; and

e) measuring an effect of the candidate compound modulator on the pharmacological activity contributed by hKv9.3 to the second potassium channel.

The instant invention is further directed to a method of identifying compounds that modulate the biological activity of a potassium channel of which hKv9.3 is an integral member, comprising:

a) providing a host cell which co-expresses at least one potassium channel subunit and a subunit comprised of the sequence substantially as depicted in SEQ ID NO:3 to produce a potassium channel; and

b) measuring the biological activity of the channel; and

c) combining a candidate compound modulator with said host cell; and

d) measuring the effect of the candidate compound modulator on the biological activity of the potassium channel.

### BRIEF DESCRIPTION OF THE FIGURES

FIG. 1 displays SEQ ID NO:1 which is a 2421 base cDNA nucleic acid sequence which encodes the novel human voltage-gated potassium channel polypeptide (hKv9.3) described herein.

FIG. 2 displays SEQ ID NO:2 which is the 1476 base translated structural coding region, ATG to TGA (Opal), of the cDNA nucleic acid sequence which encodes the novel human voltage-gated potassium channel polypeptide (hKv9.3).

FIG. 3 displays SEQ ID NO:3 which is the 491 amino acid residue sequence of the novel human voltage-gated potassium channel polypeptide (hKv9.3) described herein.

FIG. 4 shows a hydropathy plot of the amino acid residue sequence of the novel human voltage-gated potassium channel polypeptide described herein (SEQ ID NO:3) demonstrating predicted transmembrane segments. Kyte, J., Doolittle, R. F., J. Mol. Biol., 157:105 (1982); DNASTAR Inc, Madison Wis.

FIG. 5 shows SEQ ID NO:4 which is the 491 amino acid residue sequence of rKv9.3. GENBANK accession #AF029056. Patel, A. J., et al., EMBO,16(22):6615 (1997).

FIG. 6 displays a comparison alignment between the amino acid residue sequence of the novel human voltage-gated potassium channel polypeptide described herein, hKv9.3 (SEQ ID NO:3), and the amino acid residue sequences of, rKv9.3 (SEQ ID NO:4), using Clustal method with PAM250 residue weight table.

FIG. 7 shows SEQ ID NO:5 which is the pore sequence of hKv1.1 used as a query in the tblastn search. Chandy and Gutman, Handbook of Receptors and Channels, Ligand- and Voltage-gated Ion Channels, Edited by R. Alan North, CRC Press, Chp 1 (1995); Altschul, S. F., et al., J. Mol. Biol., 215:403 (1990).

FIG. 8 displays PCR primers, SEQ ID NO:6 and SEQ ID NO:7, used for hKv9.3 5′ amplification.

FIG. 9 displays PCR primers, SEQ ID NO:8 and SEQ ID NO:9, used to make full length hKv9.3 cDNA.

FIG. 10 shows SEQ ID NO: 10 which is a 2565 base structural region which encodes hKv2.1, an example human potassium channel subunit for use in methods of the invention. Ikeda, S. R., et al., Eur. J. Physiol., 422:201(1992).

FIG. 11 shows SEQ ID NO: 11 which is a voltage sensor motif used as a query in the tblastn search. Altschul, S. F., et al., J. Mol. Biol., 215:403 (1990).

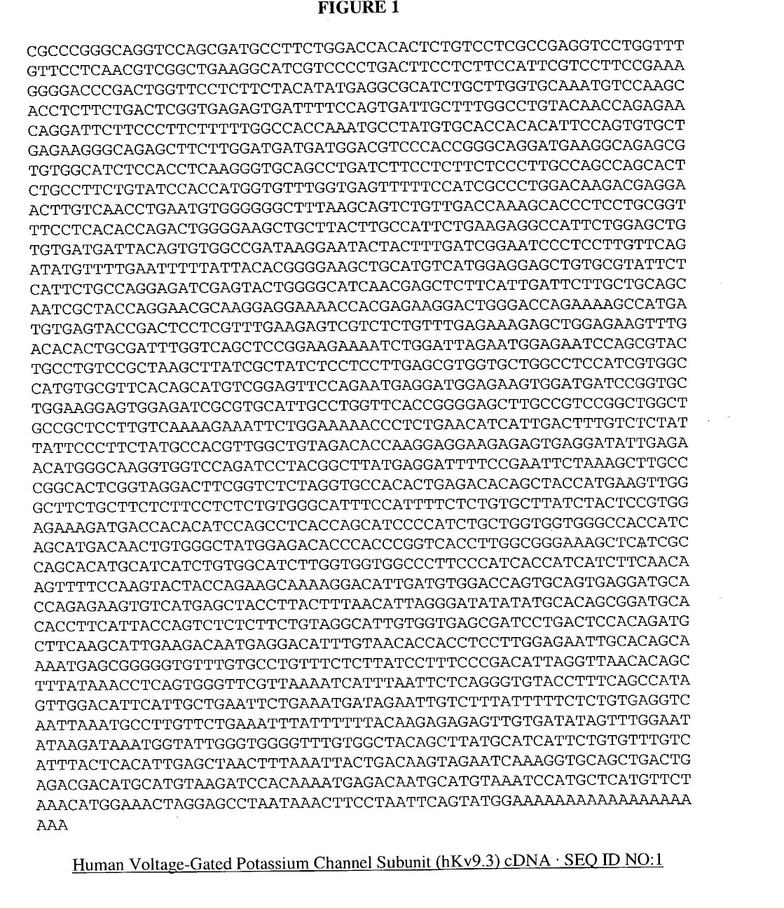

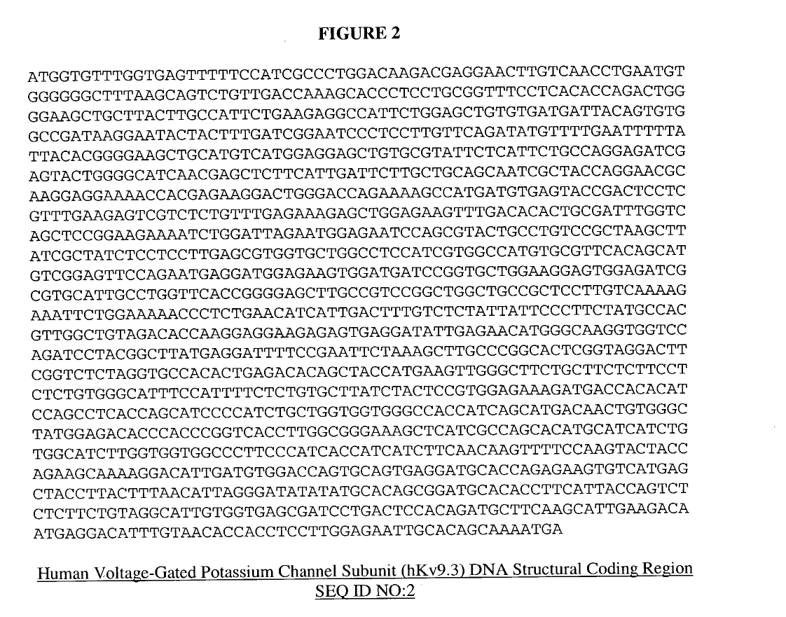

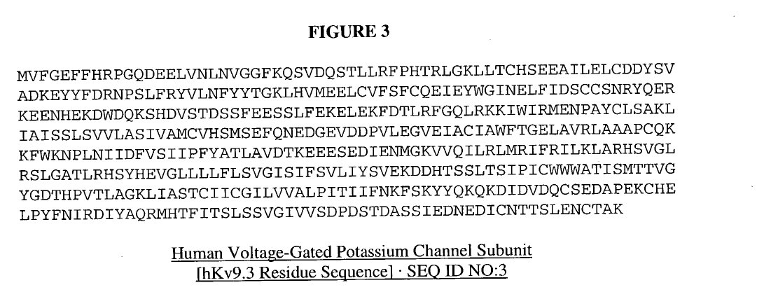

### DETAILED DESCRIPTION OF THE INVENTION

Unless defined otherwise, all technical and scientific terms used herein have the same meaning as is commonly understood by one of skill in the art to which this invention belongs. All publications and patents referred to herein are incorporated by reference.

Nucleic acid sequence as used herein refers to an oligonucleotide, nucleotide or polynucleotide sequence, and fragments or portions thereof, and to DNA or RNA of genomic or synthetic origin which may be double-stranded or single-stranded whether representing the sense or antisense strand. Similarly, amino acid and/or residue sequence as used herein refers to peptide or protein sequences or portions thereof.

Biological activity as used herein refers to the ability of a potassium channel to allow transmembrane potassium ion flow and/or transport or regulate transmembrane potassium ion flow and/or transport.

Pharmacological activity as used herein in reference to a potassium channel polypeptide or subunit refers to the ability to bind another subunit, ligand, or cofactor and/or otherwise modulate the biological activity of a potassium channel.

Purified as used herein refers to molecules, either nucleic acid or amino acid sequences, that are removed from their natural environment and isolated or separated from at least one other component with which they are naturally associated.

As used herein, a functional derivative of a potassium channel molecular structure or polypeptide disclosed herein is a compound or entity that possesses a biological or pharmacological activity (either functional or structural) that is substantially similar to SEQ ID NO:3. The term “functional derivatives” is intended to include the “fragments,” “variants,” “degenerate variants,” “analogs” and “homologues”, and to “chemical derivatives”. The term “variant” is meant to refer to a molecule substantially similar in structure and function to either an entire potassium channel molecule or to a fragment thereof. A molecule is “substantially similar” to a potassium channel polypeptide if both molecules have substantially similar structures or if both molecules possess similar pharmacological or biological activity. The term “analog” refers to a molecule substantially similar in function to either an entire native polypeptide, or to a C-terminal fragment thereof.

The term “modulation” is used herein to refer to the capacity to either enhance or inhibit a functional property of a subunit or potassium channel. The term “modulation” is also used herein to refer to the capacity to affect the biophysical activity of a cell.

Modulation or regulation of biological activity and/or pharmacological activity as used herein refers to binding, blocking, antagonization, repression, neutralization, or sequestration, of a potassium channel biomolecular structure including but not limited to the novel potassium channel poypeptide described herein; as well as up regulation or agonization or activation of a potassium channel by a compound identified by means described herein.

Modulate physiology as used herein refers to the biophysiological regulation of cells and/or tissue and the treatment of pathophysiological disorders related thereto.

‘Substantially as depicted’ as used herein refers to functional derivative proteins, variant peptides and DNA sequences that may have changes but perform substantially the same biochemical function in substantially the same way; however, ‘substantially as depicted’ as used herein also refers to dominant negative mutant versions.

Biologically active fragment or pharmacologically active fragment as used herein includes peptides which have been truncated with respect to the N- or C-termini, or both; or the corresponding 5′ or 3′ end, or both, of the corresponding polynucleotide coding region, which fragments perform substantially the same function or encode peptides which perform substantially the same function in substantially the same way. The term “biologically active” or “pharmacologically active” also refers to the activity of a homolog or analog entity having structural, regulatory or biochemical functions substantially the same as the naturally occurring entity.

Expression vector as used herein refers to nucleic acid vector constructions which have components to direct the expression of heterologous protein coding regions including coding regions of the present invention through accurate transcription and translation in host cells. Expression vectors usually contain a promoter to direct polymerases to transcribe the heterologous coding region, a cloning site at which to introduce the heterologous coding region, and usually polyadenylation signals. Expression vectors include but are not limited to plasmids, retroviral vectors, viral and synthetic vectors.

Transformed host cells as used herein refer to cells which have coding regions of the present invention stably integrated into their genome, or episomally present as replicating or nonreplicating entities in the form of linear nucleic acid or transcript or circular plasmid or vector. Transformation or transformed as used herein refers to heterologous gene expression including but not limited to transient or stable transfection systems.

Direct administration as used herein refers to the direct administration of nucleic acid constructs which encode embodiments (e.g., SEQ ID NO:3, dominant negative mutant, modulator compound molecule, antisense molecule, antibody molecule) of the present invention or fragments thereof; and the direct administration of embodiments of the present invention or fragments thereof, and the in vivo introduction of molecules of the present invention preferably via an effective eukaryotic expression vector in a suitable pharmaceutical carrier. Polynucleotides and therapeutic molecules of the present invention may also be delivered in the form of nucleic acid transcripts.

Potassium Channels

Potassium channels are regularly tetramers of related subunits. Functional potassium channels are generally formed by homomeric association of 4 identical molecules, or by heteromeric association of 2, 3, or 4 closely related K channel polypeptides. MacKinnon, R., Determination of the Subunit Stoichiometry of a Voltage-Activated Potassium Channel, Nature, 350(6315):232 (1991). A voltage gated potassium channel prominent in lymphocytes, for example, is a homotetramer of Kv1.3. Helms, L. M., et al., Biochemistry, 36(12):3737 (1997). In contrast, other voltage-gated potasssium channels are often observed as heteromultimers. Koch, R. O., et al., J. Biol. Chem., 272(44):27577 (1997). Moreover, as one example, two potassium channel gene-products (KIR and CIR) have been demonstrated to produce functional potassium currents only when simultaneously expressed. The KIR/CIR heteromultimer has been shown to constitute the native cardiac inward rectifier current with important therapeutic implications. Krapivinsky, G., et al., Nature, 374(6518):135 (1995).

Salinas et al recently described changes in electrophysiological properties of Kv2 subunits induced by rat Kv5.1 as a modulatory subunit. Salinas, M., et al., New Modulatory α Subunits for Mammalian Shab K⁺ Channels, J. Biol. Chem., 272(39): 24361 (1997). More than than 20 voltage-gated potassium channels subunits available at the time of the publication were referred to as functionally relevant, i.e., six subfamilies Kv1 (Shaker), Kv2 (Shab), Kv3 (Shaw), Kv4 (Shal), Kv7, KvLQT, and EAG (most members of these many types of subunit structures can express K⁺ channels when their cRNAs are injected into Xenopus oocytes (Hugnot, J. P., et al., Kv8.1 a New Neuronal Potassium Channel Subunit with Specific Inhibitory Properties Towards Shab and Shaw Channels, EMBO, 15(13):3322 (1996))). The authors moreover referred to electronically silent subunits which constitute subfamilies: rat Kv5.1 (IK8), Kv6.1 (K13), and kv8.1. The study showed that rat Kv5.1 is a potent regulator of channels of the Kv2 family. Rat Kv5.1 was demonstrated to inhibit the expression of both Kv2.1 and Kv2.2. Properties of Kv2 potassium currents can change in many different ways in the presence of the different modulatory subunits. Each type of cells expressing Kv2.1 or/and Kv2.2 could in principle acquire its own electrophysiological characteristics depending on the nature and stoichiometry of the associated subunits. These subunits could also serve to direct the localization of a particular Kv2.1 or Kv2.2 subunit to specific regions (cell body, dendrites, terminals, and so forth) of a given cell. Salinas, M., et al., New Modulatory α Subunits for Mammalian Shab K⁺ Channels, J. Biol. Chem., 272(39): 24361 (1997). The existence of inhibitory subunits for voltage-sensitive K⁺ channels could provide a new mode of regulation of electrical activity in cells. Hugnot, J. P., et al., EMBO, 15(13):3322 (1996)

Rat-derived Kv9.3, for example, associates with Kv2.1 to produce a novel phenotype having drastically altered biological properties including increased current, larger single channel conductance, shift in voltage-dependence for activation. Morover, immunoprecipitation studies have confirmed functional association of the two subunits. Patel, A. J., et al., EMBO J., 16(22):6615 (1997). The polypeptide has been demonstrated to associate with other potassium channel subunits to produce a dominant-negative loss of function as in certain pathological conditions shown in epilepsy, cardiac arrythmias, and/or ataxias. Salinas, M., et al., J. Biol. Chem., 272(13): 8774 (1997); Sanguinetti, M. C., et al., Neuropharmacology, 36(6):755 (1997); Biervert, C., et al., Science, 279:403 (1998). Under circumstances such as these examples, compounds or agents otherwise, that have the ability to “activate” or act as an opener of the potassium channel multimer via modulation of the pharmacological activity of one or more of the subunits and/or modulation of the channel as a whole, have significant value as potential therapeutic agents. Furthermore, pathological conditions manifested by abnormally increased potassium channel activity may be controlled by compounds or agents otherwise that have the ability to down regulate the activity of the potassium channel multimer via modulation of the pharmacological activity of one or more of the subunits and/or the biological activity of the channel as a whole.

Rat Kv9.3

Rat Kv9.3, a protein of 491 amino acids (SEQ ID NO:4) has been described by Patel, A. J., et al., EMBO,16(22):6615 (1997). The rKv9.3 Shab-like subunit in rat PA myocytes is an electrically silent subunit which associates with Kv2.1, for example, and modulates its biophysical properties. The rKv9.3 heteromultimer, unlike Kv2.1 alone, opens in the voltage range of the resting membrane potential of PA myocytes. Patel, et al., demonstrate that the activity of rKv2.1/rKv9.3 is tightly controlled by internal ATP and is reversibly inhibited by hypoxia. Metabolic regulation of the Kv2.1/rKv9.3 heteromultimer appears to play an important role in hypoxic PA vasoconstriction and in the possible development of PA hypertension. EMBO,16(22):6615 (1997).

Rat Kv9.3 belongs to a family of electrically silent potassium channels and was the first to be identified in vascular smooth muscle. Other members of this family have previously been identified in brain. The channel subunits do not express a potassium channel current by themselves, but induce profound changes in the properties of the Shab channels Kv2.1 and Kv2.2. Most interestingly, these silent subunits have the ability to create a diverse range of effects, since Kv8.1 acts as a dominant inhibitory subunit while rKv9.3 behaves as a stimulatory one. Examination of the single-channel properties of Kv2.1 and Kv2.1/rKv9.3 clearly revealed that rKv9.3 alters the single-channel conductance of Kv2.1. The pharmacological properties of Kv2.1 were altered when co-expressed with rKv9.3 in Xenopus oocytes. Both the sensitivity to TEA and 4-AP were decreased by 3- and 10-fold, respectively. In particular, rKv9.3 consistently increased Kv2.1 channel current amplitude and shifted steady-state activation towards negative values (−60 to −50 mV). Furthermore, it was reported that Kv2.1/Kv9.3 expression induced a shift in the resting membrane potential of both Xenopus oocytes and transfected COS cells (from +4 to −51 mV). The ability of rKv9.3 to ‘drag’ the Kv2.1 activation voltage threshold into the range of PA myocytes RMP suggests that the Kv2.1/rKv9.3 channel complex contributes to the setting of the RMP (−54±4 mV) and, consequently, in the setting of the resting pulmonary arterial pressure. Rat Kv9.3 also speeded up Kv2.1 activation, for instance, and dramatically slowed down deactivation. Patel, A. J., et al., EMBO,16(22):6615 (1997).

Anorexic agents such as aminorex fumarate and dex-fenfluramine (DFF) have caused an epidemic of pulmonary hypertension in Europe. These compounds have been shown to inhibit potassium currents in rat pulmonary artery myocytes and to cause pulmonary vasoconstriction. Patel, et al., confirmed these data and further demonstrate that Kv2.1 and Kv2.1/rKv9.3 complexes are similarly sensitive to the hypertensive anorexic agent, DFF. The understanding of the molecular nature of the K⁺ channels in PA myocytes will obviously have major therapeutic significance for important human pathologies such as pulmonary hypertension. The identification of openers of these novel ATP-dependent, delayed-rectifier K⁺ channels is believed to be of significant value. Opening of these channels would lead to repolarization of PA myocytes, closing of calcium channels, vasorelaxation of resistance arteries and thereby a decrease in arterial pressure. Moreover, the identification of these channels may help to define novel appetite-suppressant agents devoid of side effects on the pulmonary vasculature. Patel, A. J., et al., EMBO,16(22):6615 (1997).

Human KV9.3

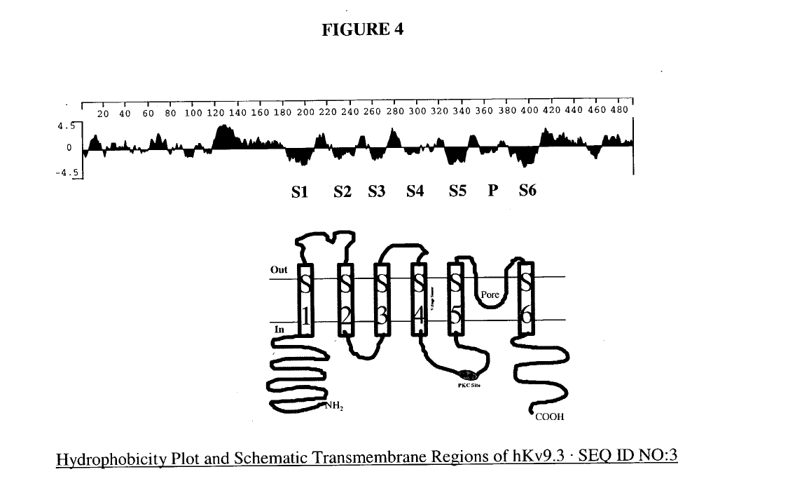

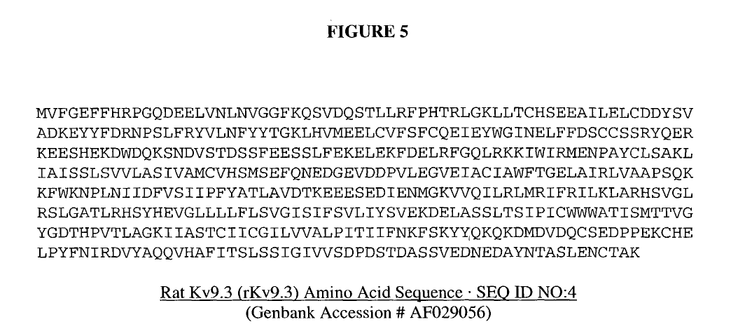

Herein disclosed is SEQ ID NO:1 which is a 2421 base cDNA nucleic acid sequence which encodes the novel human voltage-gated potassium channel subunit, hKv9.3. SEQ ID NO:2 is the 1476 base translated structural coding region, ATG to TGA (Opal), of the cDNA nucleic acid sequence which encodes the novel human voltage-gated potassium channel subunit. SEQ ID NO:3 is the 491 amino acid residue sequence of the novel human voltage-gated potassium channel subunit hKv9.3. FIG. 4 shows a hydropathy plot of the amino acid residue sequence of the novel human voltage-gated potassium channel subunit described herein (SEQ ID NO:3) demonstrating the predicted transmembrane segments. Kyte, J., Doolittle, R. F., J. Mol. Biol., 157:105 (1982); DNASTAR Inc, Madison Wis.

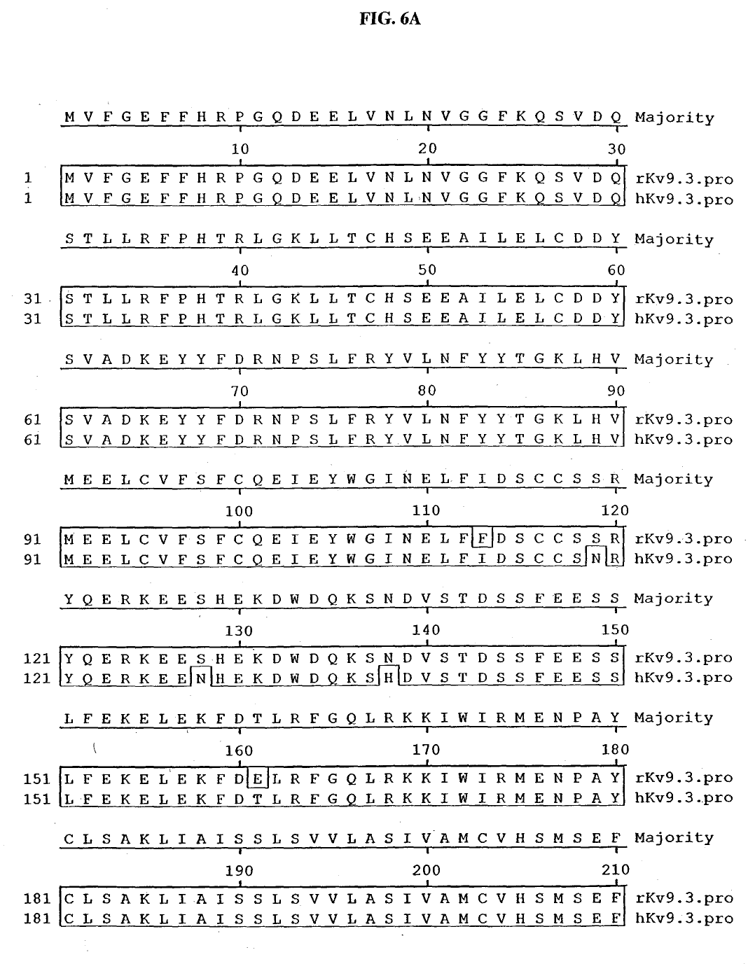

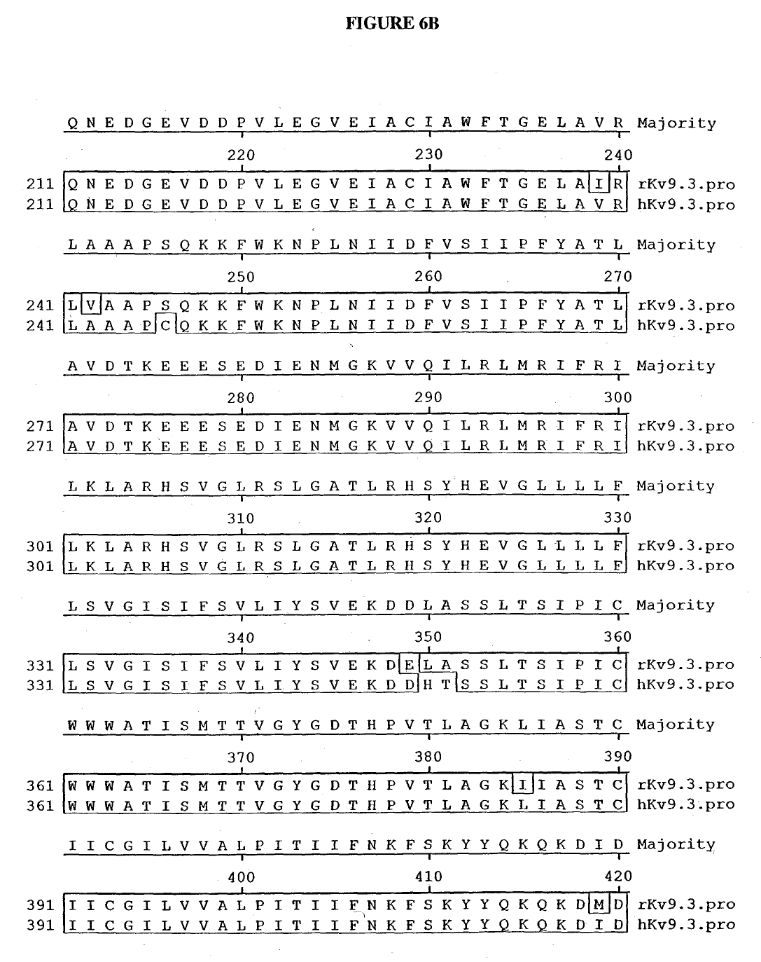

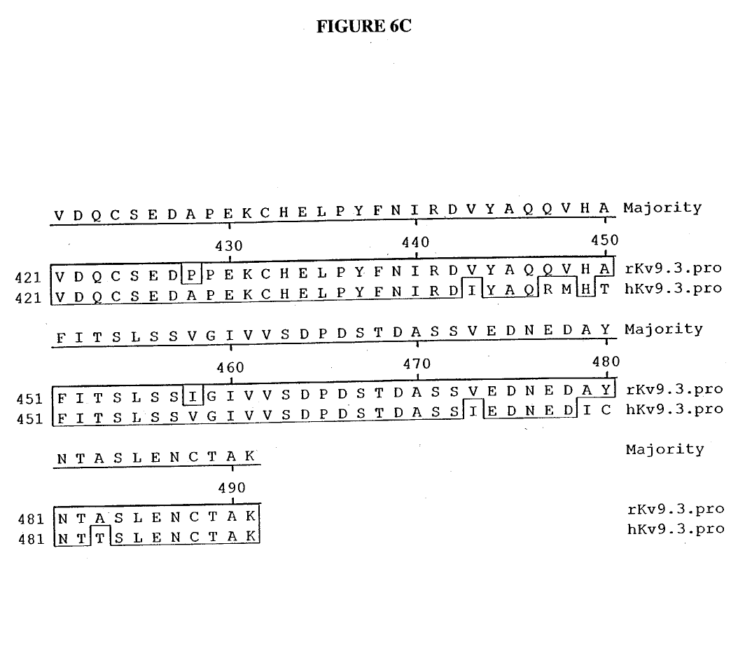

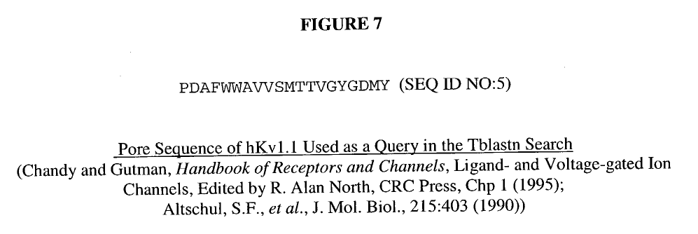

For reference and comparison: SEQ ID NO:4 is the 491 amino acid residue sequence of rKv9.3. GENBANK accession #AF029056. Patel, A. J., et al., EMBO,16(22):6615 (1997). FIG. 6 displays a comparison alignment between the amino acid residue sequence of the novel human voltage-gated potassium channel polypeptide described herein, hKv9.3 (SEQ ID NO:3), and the amino acid residue sequences of, rKv9.3 (SEQ ID NO:4), using Clustal method with PAM250 residue weight table. The 491 residue voltage-gated potassium channel subunit, human Kv9.3 (SEQ ID NO:3), shares 95% total homology at the amino acid level with the 491 amino acid residue sequence pertaining to rat-derived Kv9.3 (SEQ ID NO:4) (comparison shown in FIG. 6).

Northern blot analysis of multiple tissue RNA blots (CLONTECH, Palo Alto, Calif.), using ³²P-labeled 3′UTR (SEQ ID NO:1) sequence as a probe reveals transcription (2.5-3.00 Kb band) in heart, brain, lung, skeletal muscle and pancreas. Northern blot analysis of lung tissue from rats on days 0, 7, 14, 21 and 28 following hypoxia treatment, shows a 2-3 fold upregulation of message 14 days after hypoxia, which returns to normal on day 21. Transcript levels are unchanged in normoxic rats in this time frame.

Evidence points to the fact that human Kv9.3 (SEQ ID NO:3) described herein corresponds to a human modulatory subunit and therefore a particularly important target for therapeutic development and identification of compounds which modulate the pharmacological activity of the subunit and biological activity of a potassium channel in human tissue. Modulation, as such, is expected to be useful in treating disorders manifested by dysfunctional cells, e.g., pulmonary disorders, cardiovascular disease, neurological disorders, peripheral vascular disease, and connective tissue disorders.

In the native state, for instance, the hKv9.3 subunit is expected to exhibit a pharmacological activity toward the potassium channel of which it is an integral subunit to. Pharmacological activity as used herein refers to the inherent ability to confer a level of functionality, or ability as a ligand receptor to confer an inducible functionality or inducible level of functionality to the channel of which it is an integral subunit to. In heteromultimeric states, with regard to the novel subunit, hKv9.3, native and/or newly formed channels, in vitro and/or in vivo are expected to have biological activity clearly influenced by the pharmacological activity of hKv9.3.

Subunits that may be heteromerically associated with the novel subunit described herein are expected to be subject to pharmacological activity of the hKv9.3 subunit. The novel human voltage-gated potassium channel subunit, hKv9.3, described herein is thereby expected to confer specific heteromeric biological activity to functional potassium channels.

Compounds or agents otherwise, that have the ability to “activate” or act as an opener of the potassium channel multimer via modulation of the pharmacological activity of one or more of the subunits and/or modulation of the channel as a whole, have significant value as potential therapeutic agents. Furthermore, pathological conditions manifested by abnormally increased potassium channel activity may be controlled by compounds or agents otherwise that have the ability to down regulate the activity of the potassium channel multimer via modulation of the pharmacological activity of one or more of the subunits and/or the biological activity of the channel as a whole.

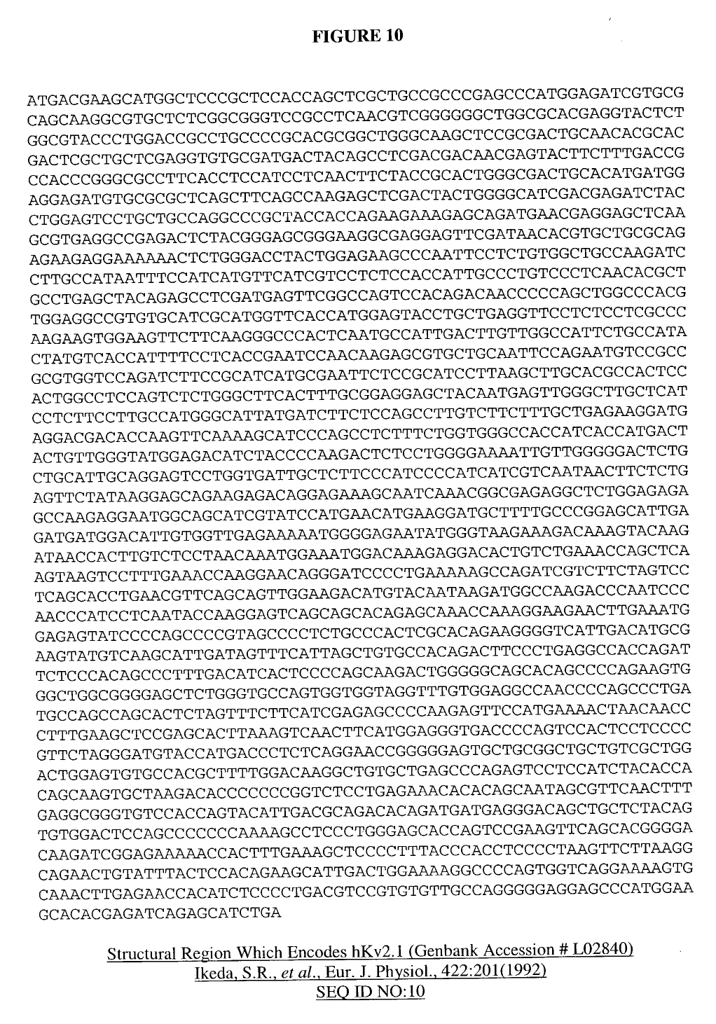

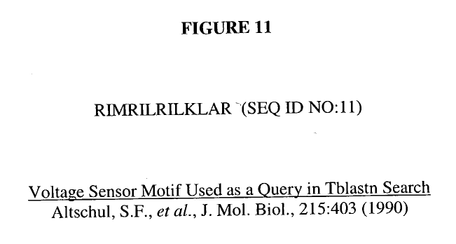

The novel human voltage-gated potassium channel subunit described herein, hKv9.3, is expected to function as a voltage-gated potassium channel alone or in conjunction with other voltage-gated potassium channel members, including but not limited to members of the Kv2 family (e.g., Kv2.1, Kv2.2, Kv2.3), KQT family, KCNQ family (recently identified or renamed as the same as the KQT family), ERG family and ELK family of six-transmembrane potassium channels. See, e.g., Chandy, K. G. and Gutman, G. A., Handbook of Receptors and Channels, CRC Press, Boca Raton, Fla. (1995); Wei, A., et al., Neuropharmacology,; 35(7):805 (1996). Kv2 subfamily members, e.g., including but not limited, to Kv2.1 (FIG. 10 shows SEQ ID NO:10 which is a 2565 base structural region which encodes hKv2.1, an example human potassium channel subunit for use in methods of the invention. Ikeda, S. R., et al., Eur. J. Physiol., 422:201(1992)), Kv2.2, Kv2.3; as well as KCNQ subfamily members, e.g., including but not limited, KCNQ2 (Bievert, C., et al., A Potassium Channel Mutation in Neonatal Human Epilepsy, Science, 279(5349):403 (1998); Singh, N. A., et al., A Novel Potassium Channel Gene, KCNQ2 is Mutated in an Inherited Epilepsy of Newborns, Nature Genetics, 18:25 (1998)) and KCNQ3 (Charlier, C., et al., A Pore Mutation in a Novel KQT-like Potassium Channel Gene in an Idiopathic Epilepsy Family, Nature Genetics, 18:53 (1998)), are examples of subunits to be used in methods of the invention.

Co-expression of potassium channel subunits is practiced for example by co-injection or co-transfection; generally co-transformation—as has been well-established in the art. Concatanated cDNA constructs that physically links different monomer constructs, in tandem, for example, are preferred in order to control the stoichiometry of expression. Co-expression may be followed by verification of functional association of the subunits via measurement of potassium channel biological activity via assays and/or immunoprecipitation of the associated subunits using specific polyclonal or monoclonal antibodies raised against one or more of the subunits. Procedures are established, for example, using polyglycine or polyglutamine linkers with engineered restriction sites to physically link in-frame, the C-terminus of one monomer to the N-terminus of the second monomer. Hurst, R. S., et al., Receptors and Channels, 3(4):263 (1995); Ishii, T. M., et al., J. Biol. Chem., 272(37):23195 (1997).

For example, using published sequences and well-known procedures, cells which express each of: hKv9.3-Kv2.1(using SEQ ID NO:10); hKv9.3-Kv2.2; hKv9.3-Kv2.3; Kv2.1-hKv9.3-Kv2.2; Kv2.1-hKv9.3-Kv2.3; Kv2.2-hKv9.3-Kv2.3; Kv2.2-hKv9.3-Kv2.3-Kv2.1; hKv9.3-KCNQ2; hKv9.3-KCNQ3; hKv9.3-KCNQ1; hKv9.3-KQT2; hKv9.3-KCNQ2-KCNQ3; hKv9.3-KCNQ2-KCNQ3-KCNQ1, can easily be constructed.

The novel human voltage-gated potassium channel homolog described herein hKv9.3 can be used to create and supplement many different potassium channel subunit compositions. Human Kv9.3 may be used to emulate the composition of native channels and/or to create novel compositions for in vivo therapeutic purposes.

Individual gene cassettes are first generated, for example, by introducing unique restriction sites at the 5′ and 3′ ends of the coding region of the gene. The restriction sites allow the cDNA to be ligated in frame and in a known orientation. Linker regions encoding 10 glutamine or glycine residues are introduced between adjacent gene cassettes. In each case, the first glutamine codon of the repeat replaces the natural stop codon in the first cassette; the last cassette has no linker. The ligation reactions generate these concatanated constructs in standard vectors which are transformed, for example, into E. coli host strains. Plasmid DNA isolated from individual colonies are analyzed by restriction digests and nucleic acid sequencing of the constructs. The constructs are then used to express functional potassium channels by in host cells, for example, heterologous mammalian or insect cells. The resulting cell-lines are used in drug screening assays including high-throughput screening (HTS) assays (e.g., Rb efflux/FLIPR, infra).

Potassium channel subunits contemplated for use in methods of the present invention include but are not limited to Kv1.1, Kv1.2, Kv1.3, Kv1.4, Kv1.5, Kv1.6, Kv1.7, Kv2.1, Kv2.2, Kv2.3, Kv3.1, Kv3.2, Kv3.3, Kv3.4, Kv4.1, Kv4.2, Kv4.3, Kv5.1, Kv6.1, Kv7.1, Kv8.1, Kv9.1, Kv9.2, Kv9.3, KQT1, KQT2, KQT3, KCNQ2, KCNQ3, ISK, HERG1, HERG2, ELK1, ELK2, all inward rectifier potassium channel subunits, and 2-pore K channels subunits. Any K channel subunit may be used in the methods of the present invention. See, e.g., Chandy, K. G. and Gutman, G. A., Handbook of Receptors and Channels, CRC Press, Boca Raton, Fla. (1995); Wei, A., et al., Neuropharmacology, 35(7):805 (1996).

Compounds which have the ability to modulate the pharmacological activity of the novel human voltage-gated potassium channel subunit described herein and/or the biological activity of potassium channels of which hKv9.3 is a subunit are therefore expected to act as therapeutic agents in pathophysiological conditions including, but not limited to, pulmonary disorders.

Variants

The present invention also encompasses variants of the human voltage-gated modulatory subunit SEQ ID NO:3. A variant substantially as depicted in SEQ ID NO:3, for instance, is one having 96% total amino acid sequence similarity to the human E3 ubiquitin protein ligase amino acid sequence (SEQ ID NO:3) or a biologically active fragment thereof. A preferred variant substantially as depicted in SEQ ID NO:3 is one which retains at least one amino acid residue which is characteristic of the human voltage-gated modulatory subunit, hKv9.3, described herein.

A “variant” of the human voltage-gated potassium channel molecule of the present invention may have an amino acid sequence that is different by one or more amine acid “substitutions”. The variant may have “conservative” changes, wherein a substituted amine acid has similar structural or chemical properties, eg, replacement of leucine with isoleucine. More rarely, a variant may have “nonconservative” changes, eg, replacement of a glycine with a tryptophan. Similar minor variations may also include amine acid deletions or insertions, or both. Guidance in determining which and how many amine acid residues may be substituted, inserted or deleted without abolishing biological or immunological activity, for instance, may be found using computer programs well known in the art, for example, DNAStar software.

The present invention relates to nucleic acid (SEQ ID NO:1 and SEQ ID NO:2) and amino acid sequences (SEQ ID NO:3) of the novel human voltage-gated potassium channel subunit and variations thereof and to the use of these sequences to identify compounds that modulate the activity of potassium channels and human pulmonary physiology.

The invention further relates to the use of the nucleic acid sequences described herein in expression systems as assays for agonists or antagonists of the potassium channel biomolecule. The invention also relates to the diagnosis, study, prevention, and treatment of disease related to a human potassium channel and/or diseases mediated by dysfunctional pulmonary tissue.

Polynucleotide sequences which encode the human voltage-gated modulatory subunit (SEQ ID NO:3) or a functionally equivalent derivative thereof may be used in accordance with the present invention which comprise deletions, insertions and/or substitutions of the SEQ ID NO:2 nucleic acid sequence. Biologically active variants of the biomolecule of the present invention may also be comprised of deletions, insertions or substitutions of SEQ ID NO:3 amino acid residues. A purified polynucleotide comprising a nucleic acid sequence encoding the polypeptide having the sequence substantially as depicted in SEQ ID NO:3 or a biologically active fragment thereof is a particularly preferred embodiment of the present invention.

Amino acid substitutions of SEQ ID NO:3 may be made, for instance, on the basis of similarity in polarity, charge, solubility, hydrophobicity, hydrophilicity, and/or the amphipathic nature of the residues as long as the pharmacological or biological activity of the potassium channel subunit is retained. For example, negatively charged amino acids include aspartic acid and glutamic acid; positively charged amino acids include lysine and arginine; and amino acids with uncharged polar head groups having similar hydrophilicity values include leucine, isoleucine, valine; glycine, alanine; asparagine, glutamine; serine, threonine phenylalanine, and tyrosine.

Nucleic acid sequences which encode the amino acid sequence of the human voltage-gated modulatory subunit described herein are of an exponential sum due to the potential substitution of degenerate codons (different codons which encode the same amino acid). The oligonucleotide sequence selected for heterologous expression is therefore preferably tailored to meet the most common characteristic tRNA codon recognition of the particular host expression system used as well known by those skilled in the art.

Suitable conservative substitutions of amino acids are known to those of skill in this art and may be made without altering the biological activity of the resulting polypeptide, regardless of the chosen method of synthesis. The phrase “conservative substitution” includes the use of a chemically derivatized residue in place of a non-derivatized residue provided that such polypeptide displays the desired binding activity. D-isomers as well as other known derivatives may also be substituted for the naturally occurring amino acids. See, e.g., U.S. Pat. No. 5,652,369, Amino Acid Derivatives, issued Jul. 29, 1997. Substitutions are preferably, although not exclusively, made in accordance with those set forth in TABLE 1 as follows:

TABLE 1

| Original residue | Conservative substitution |
|---|---|
| Ala (A) | Gly; Ser; Val; Leu; Ile; Pro |
| Arg (R) | Lys; His; Gln; Asn |
| Asn (N) | Gln; His; Lys; Arg |
| Asp (D) | Glu |
| Cys (C) | Ser |
| Gln (Q) | Asn |
| Glu (E) | Asp |
| Gly (G) | Ala; Pro |
| His (H) | Asn; Gln; Arg; Lys |
| Ile (I) | Leu; Val; Met; Ala; Phe |
| Leu (L) | Ile; Val; Met; Ala; Phe |
| Lys (K) | Arg; Gln; His; Asn |
| Met (M) | Leu; Tyr; Ile; Phe |
| Phe (F) | Met; Leu; Tyr; Val; Ile; Ala |
| Pro (P) | Ala; Gly |
| Ser (S) | Thr |
| Thr (T) | Ser |
| Trp (W) | Tyr; Phe |
| Tyr (Y) | Trp; Phe; Thr; Ser |
| Val (V) | Ile; Leu; Met; Phe; Ala |

The nucleotide sequences of the present invention may also be engineered in order to alter a coding sequence for a variety of reasons, including but not limited to, alterations which modify the cloning, processing and/or expression of the gene product. For example, mutations may be introduced using techniques which are well known in the art, eg, site-directed mutagenesis to insert new restriction sites, to alter glycosylation patterns, to change codon preference, etc.

Included within the scope of the present invention are alleles of the human potassium channel molecule of the present invention. As used herein, an “allele” or “allelic sequence” is an alternative form of the potassium channel molecule described herein. Alleles result from nucleic acid mutations and mRNA splice-variants which produce polypeptides whose structure or function may or may not be altered. Any given gene may have none, one or many allelic forms. Common mutational changes which give rise to alleles are generally ascribed to natural deletions, additions or substitutions of amino acids. Each of these types of changes may occur alone, or in combination with the others, one or more times in a given sequence.

The present invention relates, in part, to the inclusion of the polynucleotide encoding the novel potassium channel molecule in an expression vector which can be used to transform host cells or organisms. Such transgenic hosts are useful for the production of the novel pulmonary physiological molecule and variations thereof described herein.

The nucleic acid sequence also provides for the design of antisense molecules useful in downregulating, diminishing, or eliminating expression of the genomic nucleotide sequence in cells including but not limited to pulmonary tissue, and tumor or cancer cells.

The human potassium channel biomolecule of the present invention can also be used in screening assays to identify blockers, antagonists or inhibitors which bind, emulate substrate, or otherwise inactivate or compete with the biomolecule. The novel potassium channel can also be used in screening assays to identify agonists which activate the potassium channel or otherwise induce the production of or prolong the lifespan of the biomolecule in vivo or in vitro.

The invention also relates to pharmaceutical compounds and compositions comprising the human voltage-gated modulatory subunit molecule substantially as depicted in SEQ ID NO:3, or fragments thereof, antisense molecules capable of disrupting expression of the naturally occurring gene, and agonists, antibodies, antagonists or inhibitors of the native biomolecule. These compositions are useful for the prevention and/or treatment of conditions associated with pulmonary disorders.

Particularly preferred embodiments of the invention are directed to methods of screening for compounds which modulate pulmonary physiology or enhance or interfere with or inhibit the biological activity of a potassium channel of which hKv9.3 is an integral member.

Pharmacological Assays

The novel human voltage-gated potassium channel subunit described herein, hKv9.3, may be assayed for its homomeric biological activity and/or assayed for its contribution as an integral member to the biological activity of a multitude of different heteromeric potassium channels. Assays may be performed for instance wherein a hKv9.3 structural coding region, e.g., SEQ ID NO:2, is expressed in a host cell and the host cell is assayed for potassium channel activity via well-established methods including, but not limited to, those, e.g., radioisotopic Rb/K flux assays or fluroscent-based membrane potential detection assays, referenced and/or otherwise described herein.

The novel potassium channel subunit described herein may also be assayed for its ability to modulate via hKv9.3 pharmacological activity or otherwise contribute to the biological activity of a myriad of different heteromeric potassium channels, including but not limited to native physiological potassium channels. Human Kv5.1 may be therefore assayed for inherent pharmacological properties which may be useful to exploit for therapeutic purposes, i.e., administration via gene therapy or otherwise, in vivo, to control the composition and hence physiological activity of native potassium channels. Therefore, as an inherent corollary, a method of the present mention is treatment of a patient in need of such treatment for a condition which is mediated by the biological activity of a human potassium channel, comprising administration of hKv9.3 substantially as depicted in SEQ ID NO:3 or a pharmacologically active fragment thereof. Therapeutic methods of the present invention also include treatment of a patient in need of such treatment for a condition which is mediated by the biological activity of a human potassium channel, comprising administration of a nucleic acid substantially as depicted in SEQ ID NO:1 or a biologically-effective fragment thereof. Therapeutic methods of the present invention furthermore include treatment of a patient in need of such treatment for a condition which is mediated by the biological activity of a human potassium channel, comprising administration of an antisense molecule comprising the complement of the sequence substantially as depicted in SEQ ID NO:2 or a biologically-effective fragment thereof (further discussed infra).

A preferred embodiment of the present invention is a method for identifying pharmacological activity contributed by hKv9.3 to a potassium channel wherein a host cell (or cell line) is provided which expresses at least one potassium channel subunit to produce a first potassium channel (or population of channels). Potassium channel biological activity of the first potassium channel is measured and standardized. A second host cell (or parental cell-line) is transformed with a hKv9.3 structural coding region, e.g., SEQ ID NO:2 or therwise comprising a sequence substantially as depicted in SEQ ID NO:3, such that hKv9.3 is co-expressed with the first potassium channel subunit(s), in preferably an equivalent stoichiometric ratio, to produce a resulting second potassium channel (or population). Biological activity of the resulting second potassium channel is measured and compared to the biological activity of the first potassium channel thereby identifying the pharmacological activity contributed by hKv9.3 to the potassium channel.

One example of the present invention is an assay wherein a host cell (or cell line) is provided which expresses a first potassium channel subunit to produce a first homomeric tetramer potassium channel. Potassium channel biological activity of the homomeric channel is measured and standardized. A second host cell (or parental cell-line) is transformed with a hKv9.3 structural coding region, e.g., SEQ ID NO:2, such that hKv9.3 is co-expressed with the first potassium channel subunit in preferably an approximate 1:1 stoichiometric ratio to produce a resulting dimeric tetramer potassium channel (comprised of the two subunits). Biological activity of the dimeric potassium channel is measured and compared to the biological activity of the first homomeric potassium channel thereby identifying the pharmacological activity contributed by hKv9.3 to the potassium channel.

Another example of the present invention is an assay wherein a host cell (or cell line) is provided which expresses a first potassium channel subunit and a second potassium channel subunit in preferably an approximate 1:1 stoichiometric ratio to produce a first potassium channel (comprised of the two subunits). Potassium channel biological activity of the first channel is measured and standardized. A second host cell (or parental cell-line) is transformed with a hKv9.3 structural coding region, e.g., SEQ ID NO:2, such that hKv9.3 is co-expressed with the first potassium channel subunits in preferably an approximate 1:1:1 stoichiometric ratio to produce a second potassium channel. Biological activity of the resulting second potassium channel is measured and compared to the biological activity of the first potassium channel.

A further example of the present invention is an assay wherein a host cell (or cell line) is provided which expresses a first potassium channel subunit, a second potassium channel subunit, and a third potassium channel subunit in preferably an approximate 1:1:1 stoichiometric ratio to produce a first potassium channel (comprised of the three subunits). Potassium channel biological activity of the first channel is measured and standardized. A second host cell (or parental cell-line) is transformed with a hKv9.3 structural coding region, e.g., SEQ ID NO:2, such that hKv9.3 is co-expressed with the first potassium channel subunits in preferably an approximate 1:1:1:1 stoichiometric ratio to produce a resulting second channel. Biological activity of the resulting second potassium channel is measured and compared to the biological activity of the first channel.

A still further example of the present invention is an assay wherein a host cell (or cell line) is provided which expresses a first potassium channel subunit, a second potassium channel subunit, a third potassium channel subunit, and a fourth potassium channel subunit in preferably an approximate 1:1:1:1 stoichiometric ratio to produce a first potassium channel. Potassium channel biological activity of the first channel is measured and standardized. A second host cell (or parental cell-line) is transformed with a hKv9.3 structural coding region, e.g., SEQ ID NO:2, such that hKv9.3 is co-expressed with the first potassium channel subunits in preferably an approximate 1:1:1:1:1 stoichiometric ratio to produce a second channel. Biological activity of the resulting second potassium channel is measured and compared to the biological activity of the first potassium channel thereby identifying the pharmacological activity contributed by hKv9.3 to the potassium channel.

Drug Screening

Each of the methods and examples for identifying pharmacological activity contributed by hKv9.3 to a potassium channel, supra, are easily adapted to become methods of identifying compounds that modulate the pharmacological activity of hKv9.3. Generally, a first host cell is provided which expresses at least one potassium channel subunit to produce a first potassium channel. A second host cell is provided which co-expresses a potassium channel polypeptide comprising the sequence substantially as depicted in SEQ ID NO:3, along with the same at least one potassium channel subunit repoire in the first step, to produce a second potassium channel. The biological activity of the first potassium channel is compared to the biological activity of the second potassium channel (thereby identifying the pharmacological activity contributed by hKv9.3 to the potassium channel). A candidate compound modulator of hKv9.3 pharmacological activity is then combined or contacted with said second host cell. The effect of the candidate compound modulator on the pharmacological activity contributed by hKv9.3 to the second potassium channel is measured. Compounds that modulate the pharmacological activity of hKv9.3 identified in this manner are especially preferred embodiments of the invention. A further embodiment of the present invention is a method of treatment of a patient in need of such treatment for a condition which is mediated by the pharmacological activity of hKv9.3 comprising administration of a modulating compound which was identified in this manner.

Another preferred embodiment of the invention is provided wherein a structural coding region of hKv9.3 or a potassium channel polypeptide comprising the sequence substantially as depicted in SEQ ID NO:3, e.g., SEQ ID NO:2, is expressed in a host cell and canditate compounds are screened for potassium channel agonist or antagonist activity, for example, by means of radioisotopic Rb/K flux assays or fluroscent-based membrane potential detection assays as further described infra. The biological activity of a potassium channel composition, is compared to activity of the same channel or poulation in the presence of candidate compounds in order to identify compounds which modulate the biological activity of potassium channels of which hKv9.3 is an integral member.

The ability to screen compounds for the ability to modulate the biological activity of a potassium channel of which hKv9.3 is an integral member is an important object of the invention. A preferred embodiment of the present invention is provided wherein a potassium channel polypeptide having the sequence substantially as depicted in SEQ ID NO:3 (hKv9.3), e.g., SEQ ID NO:2, is expressed or co-expressed in a host cell with at least one other potassium channel subunit. The original biological activity of the resulting potassium channel is measured. Potassium channel polypeptides are preferably expressed in equivalent stoichiometric amounts, e.g., 1:1, 1:1:1, 1:1:1:1, 1:1:1:1:1. A candidate compound modulator of a potassium channel biological activity is then combined with the host-cell expressing or co-expressing a potassium channel polypeptide having the sequence substantially as depicted in SEQ ID NO:3. The effect of the candidate compound modulator on the biological activity of the potassium channel is measured and compared to the original activity of the potassium channel. Compounds that modulate the biological activity of a potassium channel of which hKv9.3 is an integral member identified in this manner are especially preferred embodiments of the invention. A further embodiment of the present invention is a method of treatment of a patient in need of such treatment for a condition which is mediated by the biological activity of a potassium channel of which hKv9.3 is an integral member comprising administration of a modulating compound which was identified in this manner.

An example of the present invention is a method of identifying compounds that modulate the biological activity of a potassium channel of which hKv9.3 is an inegral member comprising providing a host cell which co-expresses a first potassium channel subunit and a second potassium channel subunit comprised of the sequence substantially as depicted in SEQ ID NO:3 to produce a potassium channel. Potassium channel biological activity of the resulting channel is measured and standardized. A candidate compound modulator of a potassium channel biological activity is then combined with the host-cell co-expressing the potassium channel subunits. The effect of the candidate compound modulator on the biological activity of the potassium channel is measured and compared to the original activity of the potassium channel.

The method of screening compounds for the ability to modulate the biological activity of a potassium channel of which hKv9.3 substantially as depicted in SEQ ID NO:3 is an integral member is preferred wherein wherein the measured effect of the modulator on the channel is inhibition or enhancement of binding of a ligand to the channel.

The method of screening compounds for the ability to modulate the biological activity of a potassium channel of which hKv9.3 substantially as depicted in SEQ ID NO:3 is an integral member is preferred wherein wherein the effect of the modulator on the channel is inhibition or enhancement of physiological activity mediated by the potassium channel.

The method of screening compounds for the ability to modulate the biological activity of various potassium channels of which hKv9.3 substantially as depicted in SEQ ID NO:3 is an integral member is preferred wherein the sequence substantially as depicted in SEQ ID NO:3 (hKv9.3), e.g., SEQ ID NO:2, is co-expressed in a host cell with at least one other potassium channel subunit selected from the group consisting essentially of: Kv1.1, Kv1.2, Kv1.3, Kv1.4, Kv1.5, Kv1.6, Kv1.7, Kv2.1, Kv2.2, Kv2.3, Kv3.1, Kv3.2, Kv3.3, Kv3.4, Kv4.1, Kv4.2, Kv4.3, Kv5.1, Kv6.1, Kv7.1, Kv8.1, Kv9.1, Kv9.2, Kv9.3, KQT1, KQT2, KQT3, KCNQ2, KCNQ3, ISK, HERG1, HERG2, ELK1, ELK2, all inward rectifier potassium channel subunits, and 2-pore K channels subunits. Any potassium channel subunit may be used in the screening methods described herein.

The method of screening compounds is provided for the ability to modulate the biological activity of various potassium channels of which hKv9.3 substantially as depicted in SEQ ID NO:3 is an integral member.

Pharmacological Significance

The present invention relates to nucleic acid (SEQ ID NO:1 and SEQ ID NO:2) and amino acid sequences (SEQ ID NO:3) of a novel human voltage-gated potassium channel and variations thereof and to the use of these sequences to identify compounds that modulate the activity of specialized cells, particularly pulmonary tissue cells.

Potassium channel openers (PCOs), for instance, hyperpolarize smooth muscle cells by increasing membrane potassium ion permeability, thereby preventing the influx of Ca²⁺ through voltage-operated Ca²⁺ channels and thus relaxing smooth muscle. Several therapeutic areas where such a mechanism is of proven or potential utility include hypertension, male pattern baldness, asthma, and urinary incontinence. Potassium channel openers currently used in the clinic as antihypertensive agents, such as pinacidil and minoxidil, and those compounds that have proven thus far to be the most useful pharmacological tools apparently act through a mechanism involving the activation of a specific channels (ATP dependent potassium channels (K_{ATP})). Ohnmacht, C. J., et al., J. Med. Chem., 39:4592 (1996).

Compounds (e.g., small-molecules, peptides, analogs, mimetics) that modulate the biological activity of the voltage-gated potassium channel subunit described herein are contemplated for use in the treatment of a wide variey of disease conditions including, but not limited to, pulmonary vasoconstriction, pulmonary hypertension, chronic obstructive pulmonary disease (COPD), bronchitis, emphysema, asthma, hypoxia, oxidative stress, cardiovascular disease, neurological disorders, peripheral vascular disease, ischemia, stroke, cancer, proliferative disorders, autoimmunity, angiogenesis, arthritis and connective tissue disorders, shizophrenia, anxiety, depression, brain tumors, Huntington's disease, Alzheimers, Parkinson's, Lou Gehrig's, stroke, epilepsy, memory degeneration, neurodegeneration, multiple sclerosis, psychosis, urinary incontinence, diabetes, premature labour, hypertension, cardiac arrhythmias, migraine headaches, autoimmune diseases, skeletal muscle disorders, and graft rejections.

The novel human voltage-gated modulatory subunit can be used to raise diagnostic antibodies as discussed infra to detect abnormal levels of the biomoleule in vivo. Therefore, in accordance with yet a further aspect of the present invention, there are provided antibodies against the potassium channel poypeptide which may used as part of various diagnostic assays for detecting physiological disorders.

The present invention relates to a screening assay for identifying molecules which have a modulating effect, e.g., compounds including but not limited to agonists and antagonists, on the biological activity of potassium channel which comprises the voltage-gated potassium channel subunit of the present invention.

Particularly preferred embodiments of the present invention are host cells transformed with a purified polynucleotide comprising a nucleic acid sequence encoding the polypeptide having the sequence substantially as depicted in SEQ ID NO:3 or a biologically active fragment thereof. Cells of this type or preparations made from them may be used to screen for pharmacologically active modulators of the novel potassium channel subunit activity using methods which are well known in the art. See, e.g., U.S. Pat. No. 5,397,702, Assay For and Treatment of Autoimmune Diseases, issued Mar. 14, 1995; U.S. Pat. No. 5,637,470, Screening array using cells expressing recombinant alpha and beta subunits of the mammalian large-conductance (maxi-K) potassium channel, issued Jun. 10, 1997; U.S. Pat. No. 5,607,843, Nucleic Acid Sequence Encoding Apamin Binding Protein, issued Mar. 4, 1997; Published PCT international application WO9603415A1, Human Potassium Channel; and U.S. Pat. No. 5,602,169, 3-substituted oxindole derivatives as potassium channel modulators, issued Feb. 11, 1997, each of which are herein incorporated by reference.

Generally Acceptable Vectors

In accordance with the present invention, polynucleotide sequences which encode the novel potassium channel subunit, fragments of the polypeptide, fusion proteins, or functional equivalents thereof may be used in recombinant DNA molecules that direct the expression of the modulatory biomolecule in appropriate host cells. Due to the inherent degeneracy of the genetic code, other DNA sequences which encode substantially the same or a functionally equivalent amino acid sequence, may be used to clone and express the novel modulatory biomolecule. As will be understood by those of skill in the art, it may be advantageous to produce the subunit encoding nucleotide sequences possessing non-naturally occurring codons.

Specific initiation signals may also be required for efficient translation of a potassium channel subunit nucleic acid sequence. These signals include the ATG initiation codon and adjacent sequences. In cases where the novel nucleic acid sequence, e.g., SEQ ID NO:2, its initiation codon and upstream sequences are inserted into the appropriate expression vector, no additional translational control signals may be needed. However, in cases where only coding sequence, or a portion thereof, is inserted, exogenous transcriptional control signals including the ATG initiation codon must be provided. Furthermore, the initiation codon must be in the correct reading frame to ensure transcription of the entire insert. Exogenous transcriptional elements and initiation codons can be of various origins, both natural and synthetic.

Nucleic acid sequences, e.g., SEQ ID NO:2, may be recombinantly expressed to produce a pharmacologically active potassium channel biomolecule by molecular cloning into an expression vector containing a suitable promoter and other appropriate transcription regulatory elements, and transferred into prokaryotic or eukaryotic host cells to produce the novel polypeptide. Techniques for such manipulations are, for instance, fully described in Sambrook, J., et al., Molecular Cloning Second Edition, Cold Spring Harbor Press (1990), and are well known in the art.

Expression vectors are described herein as DNA sequences for the transcription of cloned copies of genes and the translation of their mRNAs in an appropriate host cell. Such vectors can be used to express nucleic acid sequences in a variety of hosts such as bacteria, bluegreen algae, plant cells, insect cells, fungal cells, human, and animal cells. Specifically designed vectors allow the shuttling of DNA between hosts such as bacteria-yeast, or bacteria-animal cells, or bacteria-fungal cells, or bacteria-invertebrate cells.

A variety of mammalian expression vectors may be used to express the recombinant modulatory molecule and variations thereof disclosed herein in mammalian cells. Commercially available mammalian expression vectors which are suitable for recombinant expression, include but are not limited to, pcDNA3 (Invitrogen), pMClneo (Stratagene), pXT1 (Stratagene), pSG5 (Stratagene), EBO-pSV2-neo (ATCC 37593) pBPV-1 (8-2) (ATCC 37110), pdBPV-MMTneo (342-12) (ATCC 37224), pRSVgpt (ATCC 37199), pRSVneo (ATCC 37198), pSV2-dhfr (ATCC 37146), pUCTag (ATCC 37460), and IZD35 (ATCC 37565), pLXIN and pSIR (CLONTECH), pIRES-EGFP (CLONTECH). INVITROGEN corporation provides a wide variety of commercially available mammalian expression vector/systems which can be effectively used with the present invention. INVITROGEN, Carlsbad, Calif. See, also, PHARMINGEN products, vectors and systems, San Diego, Calif.

Baculoviral expression systems may also be used with the present invention to produce high yields of active protein. Vectors such as the CLONETECH, BacPak™ Baculovirus expression system and protocols are preferred which are commercially available. CLONTECH, Palo Alto, Calif. Miller, L. K., et al., Curr. Op. Genet. Dev. 3:97 (1993); O'Reilly, D. R., et al., Baculovirus Expression Vectors: A Laboratory Manual, 127. Vectors such as the INVITROGEN, MaxBac™ Baculovirus expression system, insect cells, and protocols are also preferred which are commercially available. INVITROGEN, Carlsbad, Calif. See EXAMPLE IV.

### Example Host Cells

Host cells transformed with a nucleotide sequence which encodes a potassium channel subunit of the present invention may be cultured under conditions suitable for the expression and recovery of the encoded protein from cell culture. Particularly preferred embodiments of the present invention are host cells transformed with a purified polynucleotide comprising a nucleic acid sequence encoding the polypeptide having the sequence substantially as depicted in SEQ ID NO:3 or a biologically active fragment thereof. Cells of this type or preparations made from them may be used to screen for pharmacologically active modulators of the activity of the voltage-gated subunit. Modulators thus identified will be used for the regulation of pulmonary physiology.

Eukaryotic recombinant host cells are especially preferred as otherwise descibed herein or are well known to those skilled in the art. Examples include but are not limited to yeast, mammalian cells including but not limited to cell lines of human, bovine, porcine, monkey and rodent origin, and insect cells including but not limited to Drosophila and silkworm derived cell lines. Cell lines derived from mammalian species which may be suitable and which are commercially available, include but are not limited to, L cells L-M(TK-) (ATCC CCL 1.3), L cells L-M (ATCC CCL 1.2), 293 (ATCC CRL 1573), Raji (ATCC CCL 86), CV-1 (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C1271 (ATCC CRL 1616), BS-C-1 (ATCC CCL 26) and MRC-5 (ATCC CCL 171).

STRATAGENE, hNT Neurons, human neurons are commercially available, for instance, for transfection, the study of ion channel genes, and drug screening, La Jolla, Calif. Pleasure, S. J., et al., J. Neuroscience, 12:1802 (1992); J. Neuroscience, 35:585 (1993); Hiraka, G., et al., J. Virol., 65:2732 (1991); Wertkin, R., et al., PNAS, 90:9513 (1993); Younkin, D. P., et al., PNAS, 90:2174 (1993).

The expression vector may be introduced into host cells expressing the voltage-gated potassium channel polypeptide via any one of a number of techniques including but not limited to transformation, transfection, lipofection, protoplast fusion, and electroporation. Commercially available kits applicable for use with the present invention for hererologous expression, including well-characterized vectors, transfection reagents and conditions, and cell culture materials are well-established and readily available. CLONTECH, Palo Alto, Calif.; INVITROGEN, Carlsbad, Calif.; PHARMINGEN, San Diego, Calif.; STRATAGENE, LaJolla, Calif. The expression vector-containing cells are clonally propagated and individually analyzed to determine the level of the novel potassium channel biomolecule production. Identification of host cell clones which express the polypeptide may be performed by several means, including but not limited to immunological reactivity with antibodies described herein, and/or the presence of host cell-associated specific potassium channel activity, and/or the ability to covalently cross-link specific substrate to the poypeptide with the bifunctional cross-linking reagent disuccinimidyl suberate or similar cross-linking reagents.

The modulatory subunit biomolecule of the present invention may also be expressed as a recombinant protein with one or more additional polypeptide domains added to facilitate protein purification. Such purification facilitating domains include, but are not limited to, metal chelating peptides such as histidine-tryptophan modules that allow purification on immobilized metals (Porath, J., Protein Exp. Purif. 3:263 (1992)), protein A domains that allow purification on immobilized immunoglobulin, and the domain utilized in the FLAGS extension/affinity purification system (Immunex Corp, Seattle Wash.). The inclusion of a cleavable linker sequences such as Factor XA or enterokinase (Invitrogen, San Diego Calif.) between the purification domain and the potassium channel coding region is useful to facilitate purification.

Systems such as the CLONTECH, TALON™ nondenaturing protein purification kit for purifying 6×His-tagged proteins under native conditions and protocols are preferred which are commercially available. CLONTECH, Palo Alto, Calif.

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation and acylation. Post-translational processing which cleaves a nascent form of the protein may also be important for correct insertion, folding and/or function. Different host cells such as CHO, HeLa, MDCK, 293, WI38, NIH-3T3, HEK293 etc., have specific cellular machinery and characteristic mechanisms for such post-translational activities and may be chosen to ensure the correct modification and processing of the introduced, foreign protein.

For long-term, high-yield production of recombinant proteins, stable expression is preferred. For example, cell lines which stably express the novel potassium channel poypeptide may be transformed using expression vectors which contain viral origins of replication or endogenous expression elements and a selectable marker gene. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clumps of stably transformed cells can be proliferated using tissue culture techniques appropriate to the cell type.

The human biomolecule described herein can be produced in the yeast S. cerevisiae following the insertion of the optimal cDNA cistron into expression vectors designed to direct the intracellular or extracellular expression of the heterologous protein. In the case of intracellular expression, vectors such as EmBLyex4 or the like are ligated to the beta subunit cistron. See, e.g., Rinas, U., et al., Biotechnology, 8:543 (1990); Horowitz, B., et al., J. Biol. Chem., 265:4189 (1989). For extracellular expression, a potassium channel coding region, e.g., SEQ ID NO:2, is ligated into yeast expression vectors which may employ any of a series of well-characterized secretion signals. Levels of the expressed potassium channel molecule are determined by the assays described herein.

A variety of protocols for detecting and measuring the expression of the potassium channel modulatory subunit, using either polyclonal or monoclonal antibodies specific for the protein are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes may be employed. Well known competitive binding techniques may also be employed. See, e.g., Hampton, R., et al. (1990), Serological Methods—a Laboratory Manual, APS Press, St Paul Minn.; Maddox, D. E., et al., J. Exp. Med. 158:1211.

In Vitro Synthesis of Capped mRNA

The full-length cDNA (SEQ ID NO:1), for example, may be used in standard procedures to synthesize biologically active mRNA for functional expression in heterologous cells, in Xenopus oocytes, for instance, or in various types of mammalian cells including human neurons. See, e.g., Goldin A, Methods Enzymol, 207:279 (1992); STRATAGENE, hNT Neurons, commercially available for the study of ion channel genes, La Jolla, Calif.

The coding region for the novel potassium channel described herein (e.g., a region which comprises SEQ ID NO:2) which encodes the polypeptide having a bio-active sequence substantially as depicted in SEQ ID NO:3 or an active fragment thereof may be cloned 3′, for example, to a bacteriophage promoter, e.g., an SP6, T7 or T3 promoter. The PGEM vectors from Promega, Madison, Wis., are examples of preferred vectors which may be used with the present invention. Standard vectors known in the art, such as pSP64T or pBSTA, which enhance stabililty of the message and increase specific expression in Xenopus oocytes, are especially preferred. Kreig and Melton, Nucleic Acids Res., 12:7057 (1984). These particular preferred vectors contain the Xenopus beta-globin 5′ and 3′ untranslated mRNA regions flanking the 5′end and a poly-A tail on the 3′ end of the gene.

A plasmid vector DNA construct which contains an insert which encodes the novel potassium channel subunit described herein or an active fragment thereof (e.g., SEQ ID NO: 1, or a region which comprises SEQ ID NO:2, or a truncated version thereof) may be then cut with a restriction enzyme to linearize the construct 3′ to the structural coding region. The linearized vector should be extracted with phenol-chloroform-isoamyl alcohol, precipitated with ethanol and re-suspended in RNAse-free water for use as a transcription template. The transcription reaction, for example, may be carried out as described infra to synthesize biologically active mRNA for subsequent in vivo or in vitro translation by methods which are well-known in the art. The mMessage mMachine™ kit, AMBION, Austin, Tex., is especially preferred for synthesizing biologically active mRNA. Alternately, the mRNA transcripts may be used as probes for analysis of tissue distribution ny Northern analyses and or RNAse protection assays.

mRNA SYNTHESIS
PGEM, Promega, Madison, WI

| | | |
|---|---|---|
| 10x SP6/T7 buffer | 5 | ul |
| 10X ATP, CTP, UTP (5 mM each) | 5 | ul |
| 10x GTP (5 mM) | 1 | ul |
| 10x GpppG (Cap; 5 mM) | 5 | ul |
| DTT (1M) | 0.5 | ul |
| Rnase inhibitor (40 U/ul) | 1.25 | ul |
| Water | 27 | ul |
| Linearized DNA (1 ug/ul) | 1-5 | ul |
| SP6 or T7 RNA polymerase (20 U/ul) | 1-3 | ul |
| TOTAL REACTION VOLUME | 50 | ul |

Incubate at 37 degrees for 1-2 hours. Add 1 ul of RNAse-free Dnase to degrade template DNA. Digest for 10 minutes. Add 75 ul of water. Extract with phenol/CHCl3. Ethanol precipitate RNA. Store in aliqots at −70 degrees.

Functional Expression

Biologically active mRNA can be introduced into heterologous cells for functional expression and analyses by methods well-known in the art. Synthetic mRNA from example constructs described supra, for example, may be injected into Xenopus oocytes for functional expression and analyses. Goldin, A., Methods Enzyml.,207:266, (1992). Hererologous potassium channels may be examined using standard two-electrode voltage clamp techniques. See, e.g., Stuhmer, W., Methods in Enzymol., 207:319, (1992); Kohler, et al., Science, 273:1709 (1996). Potassium concentrations inside the cell may be altered, for example, by adding a potassium-ionophore, co-expression with a receptor that causes a rise in intracellular potassium. Potassium concentrations may alternately be altered by pulling inside-out patches and changing potassium concentrations in the bath medium. E.g., Grissmer, S., et al., Calcium-activated Potassium Channels in Resting and Activated Human T Lymphocytes, J. Gen. Physiol, 102:601 (1993). Standard biophysical parameters, such as activation, potassium dependence, single-channel conductance, inactivation, tail currents, potassium selectivity, and thorough pharmacology of various K channel blockers including TEA, Aparmin, and others may also be tested. Grissmer, S., et al., Calcium-activated Potassium Channels in Resting and Activated Human T Lymphocytes, J. Gen. Physiol, 102:601 (1993).

The reader is referred to the heterologous expression and purification methods of Salinas, M., et al., J. Biol. Chem., 272(39): 24371 (1997), and Hugnot, J. P., et al., EMBO, 15(13):3322 (1996), which are herein incorporated by reference.

Alternatively, cRNA (synthetic mRNA from a cDNA construct) can be introduced into heterologous mammalian cells, for example, RBL cells (ATCC # CRL 1378), and 293 cells (ATCC # CRL 1573), may be transformed using standard art-methods. For example, the Eppendorf microinjection system may be used (Micromanipulator 5171 and Transjector 5242). Transformed cells may be analysed for K⁺ currents about 4 hours later using patch-clamp techniques which are well-documented. E.g., Ikeda, et al., Pfueg. Arch. Eur. J. Physiol., 422:201 (1992); Grissmer, et al., J. Gen. Physiol., 102:601 (1993).

Over-Expression of the Novel Subunit in Cell-Lines

Transient and/or stable eucaryotic transfectant cells comprised of the coding region(s) described herein are contemplated for high-level expression of the novel potassium channel.

Eucaryotic transfectants are preferred embodiments of the present invention for employment in studies for the identification molecules which modulate the novel subunit described herein in vivo. HEK cells are preferred.

Transient expression of coding regions for the voltage-gated potassium channel polypeptide can be achieved by straight transfection into mammalian cells, by standard techniques. Omari, K. et al., J. Physiol., 499:369, (1997); Panyi, G. et al., J. Gen. Physiol., 107(3):409 (1996). High level transient expression may be achieved using standard viral systems, e.g., Baculovirus, Adenovirus, or Vaccinia virus. Channel numbers resulting from these systems are typically 5-500K per cell. Kamb, A., Methods Enzymol. 207:423 (1992); Sun, T. et al., Biochemistry, 33(33):9992 (1994); Spencer, R. H., et al., J. Biol. Chem., 272:2389 (1997).

Stable transfection of heterologous cells using sequences which encode the potassium channel subunit described herein (SEQ ID NO:3) or pharmacologically active variations or fragments thereof can be generated using, for example, NIH-3t3, L929, COS, HEK, or CHO cells. See, e.g., EMBO, 11(6):2033 (1992); Grissmer, et al., Mol. Pharm., 45:1227 (1994).

A preferred vector for use with the present invention is pcDNA/Neo, which is commercially available from INVITROGEN, Carlsbad, Calif.

Cells, NIH-3t3, for example, are grown to 50% confluency in 60 mm plates (media, and conditions are according to requirements of the particular cell line) and transfected with 5 ug of pure DNA comprising a coding region for the potassium channel subunit, e.g. SEQ ID NO:2, in pcDNA/Neo using the Lipofection reagent, as described by the supplier (LIFE TECHNOLOGIES Gibco BRL, Bethesda, Md.). After transfection, the cells are incubated at 37° C., conditions for 3 days in medium with 10% FCS. Cells are trypsinized seeded onto 100 mm dishes, and then selected with 300 ug/ml of G418 (Neomycin). Only cells that have stable integration of the heterologous coding region will grow in the presence of G418, which is confered by the Neomycin-resistance gene in the plasmid. Isolated clones are processed for 2-3 rounds of purification and subjected to patch-clamp analysis for K⁺ currents.

Since the novel gene, e.g., SEQ ID NO:1 is highly expressed in differentiated cells, and since potassium channels are potently blocked by particular ligands including but not limited to tetraethylammonium chloride (TEA), 4-aminopyridine (4-AP, as well as 2-AP and 3-AP), 3,4- and 2,3-diaminopyridine, BaCl₂, CsCl, strychnine, phencyclidine, pyridostigmine, 9-aminoacridine, DuP-996 (3,3-bis (4-pyridinylmethyl)-1-phenylindolin-2-one; linopiridine), clofilium, quinidine, aminoquinolines and quinine, various cell-lines that heterologously over-expresses the novel channel structural coding regions described herein can be used in radio-labeled binding assays to screen for molecules which block potassium channels of which hKv9.3 is an integral member, using a radiolabelled ligand, as a measurable displacement entity in a binding assay or competitive binding assay. Peptide toxins including but not limited to stichodactylotoxin, apamin, charybdotoxin, kaliotoxin, and margotoxin may also be used as ligands in the binding assays described herein. See, for instance, EXAMPLE IX.

Ligand-Binding Assay for High-Throughput Screening for Modulators

Embodiments of the present invention are cell-lines that heterologously over-expresses the subunit coding region described herein (e.g., SEQ ID NO: 1 or an active truncated version thereof or a chimeric fusion) and their use in assays.

For example, a radio-labeled binding assay using a radiolabelled ligand may be used as previously decribed by Hill, R. J., Mol. Pharm., 48:98 (1995), and Deutsch, C., et al., J. Biol. Chem., 266:3668 (1991). Membrane preparations of cell-lines which over-express the subunit described herein, e.g., SEQ ID NO:3, and at least one other potassium channel subunit, are made by homogenizing the cells using a Polytron for 25 seconds at 13,000 RPM and spun at low speed (100 g) for 2 minutes. The supernatant is spun at high speed (50,000 g) for 10 minutes. The pellet is suspended in 1 ml of assay buffer (5 mM Nacl, 5 mM KCL, 10 mM HEPES, 6 mM glucose, pH 8.4) and diluted to 50 ug/ml.

To each well of a 96-well microtiter plate, 130 ul of assay buffer is added, as well as 20 ul of test molecule compound (test drug, for instance, a small molecule, peptide, analog or mimetic compound)/control assay buffer/non-specific (10 nM cold ligand) (not-labelled) 50 ul of membranes from cells which over-express a potassium channel at 50 ug/ml and 50 ul of radioligand (25 pM; NEN, 2200 Ci/mmol) are incubated for 20 minutes at 21° C. with mixing. Bound radiolabeled ligand is separated from free radiolabeled ligand in solution by filtering over pre-soaked GF/C Unifilters (Packard Instruments) and washing rapidly in ice-cold wash buffer. Upon drying, the filter plates are scintillation counted. Data from saturation experiments are subject to Scatchard analysis and linear regression. Deutsch, et al., J. Biol. Chem., 266:3668 (1991). Compounds that compete with the radio-labeled ligand for binding are identified which produce a reduction in specific counts.

In another embodiment, a scintillation proximity assay (SPA) that eliminates the need for filters, can be easily adapted for high throuput screening (HTS) assays. Hoffman, R., et al., Anal. Biochem., 20370 (1992); Kienuis, C. B. M., et al., J. Recept. Res., 12:389 (1992).

⁸⁶Rb (or ⁴²K) Efflux Assays in Cells Which Co-Express the Novel Potassium Channel Polypeptide and and at Least One Other Potassium Channel Subunit

Cells are grown to confluence in DMEM+10% FCS in six well plates. Cells are incubated in HEPES buffer solution (in mM: NaCl 137, KCl 4.7, MgSO₄ 0.6, CaCl₂ 1.8, HEPES 20, Glucose (7.7) containing ⁸⁶Rb (5 mCi/ml) overnight in a incubator at 37° C. At the end of incubation HEPES containing ⁸⁶Rb is aspirated and cells are washed with tracer free HEPES to remove extracellular ⁸⁶Rb. Cells are incubated with tracer free HEPES for 3 minutes and at the end of 3 minutes HEPES is aspirated and fresh HEPES is added to the cells. After the basal release of ⁸⁶Rb reaches a steady state the cells are exposed to HEPES containing higher KCl (20 mM) concentration for 6 minutes (3 min.×2) followed by another solution containing 20 mM KCl and compound of choice for 12 minutes (3 min.×4). At the end of the procedure 500 mL of NaOH is added to the wells and cells are scraped. The control cells are treated with HEPES containing 20 mM KCl and vehicle (DMSO) for 12 min. At the end of experiment radioactivity in aspirated solutions and the cells are counted in a gamma counter. The efflux rate constants (k min⁻¹), which represents the radioactivity released per minute expressed as a percentage of the radioactivity remaining in the tissue, is calculated. Results are expressed as maximum % increase in efflux rate constant obtained as a difference between the peak increase in rate constant and the average rate constant during 10 minutes before candidate compound application. See also EXAMPLE X.

Alternately, a cell-line (eg: HEK 293, CHO, COS or SF9) heterologously co-expressing a novel nucleic acid sequence described herein, e.g., SEQ ID NO:2, to yield a pharmacologically active peptide SEQ ID NO:3, is incubated with ⁸⁶RbCl (10 mCi/ml) for 18 hours at 37 degrees in 5% CO₂. Cells are washed in low-K⁺ HBSS medium and resuspended at 5.5×10⁶ cells/ml in low-K⁺ HBSS. Fifty ul of the cell suspension is incubated for 15 minutes at ambient temperature with 10 ul of candidate compound in a well of Millipore Multi-screen 96-well 0.65 um filtration plate (# MADV-N6550). The mixture is then incubated for 20 minutes at the same temperature with 125 ul of 140 mM K⁺-HBSS added to initiate Rb efflux via membrane depolarization and filtered with a TV-1 vacuum transfer manifold (Pall Trinity Micro, Cortland, N.Y.) into standard 96-well microtiter plates; 100 ul of the filtrate is removed and counted in a Beckman liquid scintillation counter (Palo Alto, Calif.) for one minute. Compounds that inhibit the potassium channel activity will have reduced Rb efflux. See also Example X.

Laser-Based Fluorescent Imaging Plate Reader (FLIPR)

Since the novel potassium channel subunit described herein has all the molecular features of a voltage-gated potassium channel, a fluroscent assay that detects changes in membrane potential in cells functionally co-expressing the novel biomolecule and at least one other potassium channel subunit is another method for rapid screening of small molecules that modulate hKv9.3 pharmacological activity or the biological activity of a potassium channel of which hKv9.3 is an integral member. Such an assay may be set up using fluorescent membrane potential sensing dyes such as Bis-oxynol. Brauner, et al., Biochimica et Biophysica Acta, 771:208 (1984). Cells co-expresssing SEQ ID NO:2, for example, and at least one other potassium channel subunit are incubated with the dye and candidate compound modulator. Membrane depolarization is triggered by increasing potassium concentration external to the cells. Normally, the dye partition into the membrane upon sensing depolarization, leading to a change in fluorescence. Cells co-expressing SEQ ID NO:2 are expected to open in response to depolarization and efflux potassium ions and cause a counteractive hyperpolarization and reduced efficiency of the dye to change fluorescence. The presence of ligands, either agonists or antagonists of the novel potassium channel subunit would modulate changes in fluorescence which are detected optically. The FLIPR instrument is now commercially available through Molecular Devices Inc., Sunnyvale, Calif., and can be adapted to a very rapid and sensitive 96-well screening assay. See EXAMPLE XI.

Various Screening Assays

The present invention is also directed to methods for screening for compounds which modulate the pharmacological activity of the voltage-gated modulatory subunit and/or the biological activity of a potassium channel of which hKv9.3 is an integral member in vivo. Compounds which modulate these activities may be DNA, RNA, peptides, proteins, or non-proteinaceous organic molecules. Compounds may modulate the activity by increasing or attenuating the expression of DNA or RNA which encode the subunit, or may antagonize or agonize the activity of the Kv5.1 subunit itself. Compounds that modulate the expression of DNA or RNA encoding the subunit or the function of the polypeptide may be detected by a variety of assays. The assay may be a simple “yes/no” assay to determine whether there is a change in expression or function. The assay may be made quantitative by comparing the expression or function of a test sample with the levels of expression or function in a standard sample.

The human voltage-gated potassium channel subunit described herein, its immunogenic fragments or oligopeptides can be used for screening therapeutic compounds in any of a variety of drug screening techniques. The fragment employed in such a test may be free in solution, affixed to a solid support, borne on a cell surface, or located intracellularly. The abolition of activity or the formation of binding complexes, between the potassium channel biomolecule and the agent being tested, may be measured. Accordingly, the present invention provides a method for screening a plurality of compounds for specific binding affinity with the potassium channel polypeptide or a fragment thereof, comprising providing a plurality of compounds; combining a polypeptide of the present invention or a fragment thereof with each of a plurality of compounds for a time sufficient to allow binding under suitable conditions; and detecting binding of the subunit, or fragment thereof, to each of the plurality of compounds, thereby identifying the compounds which specifically bind the human voltage-gated biomolecule.

In order to purify a potassium channel poypeptide to measure a binding activity, the source may be a whole cell lysate, prepared by one to three freeze-thaw cycles in the presence of standard protease inhibitors. The potassium channel may be partially or completely purified by standard protein purification methods. The potassium channel polypeptides described herein may be purified by affinity chromatography using specific antibody described herein or by ligands specific for an epitope tag engineered into the recombinant molecule moreover described herein. The preparation may then be assayed for binding activity as described.

Purified polypeptides comprising the amino acid sequence substantially as depicted in SEQ ID NO:3 are especially preferred embodiments of the present invention. A preferred embodiment of the present invention is a method of screening a plurality of compounds for binding affinity with the human voltage-gated potassium channel subunit (SEQ ID NO:3) by providing a plurality of compounds and combining the compounds with the human voltage-gated potassium channel subunit for a time sufficient for the compound to bind the subunit; and detecting and recovering the compound which binds the human voltage-gated potassium channel subunit. Compounds that modulate the pharmacological activity of hKv9.3 identified in this manner are especially preferred embodiments of the invention. A further embodiment of the present invention is a method of treatment of a patient in need of such treatment for a condition which is mediated by the biological activity of a potassium channel of which hKv9.3 is an integral member comprising administration of a modulating compound which was identified in this manner. A further embodiment of the present invention is a method of treatment of a patient in need of such treatment for a condition which is mediated by the pharmacological activity of hKv9.3 comprising administration of a modulating compound which was identified in this manner.

Compounds which are identified generally according to methods described, referenced, and contemplated herein that modulate the activity of hKv9.3 or a potassium channel of which Kv9.3 is an integral member (preferably regulate physiology and/or regulate pulmonary physiology) are especially preferred embodiments of the present invention.

Yeast 2-Hybrid System

In another embodiment of the invention, a nuleic acid sequence which encodes a potassium channel molecule substantially as depicted in SEQ ID NO:3 or a pharmacologically active fragment thereof may be ligated to a heterologous sequence to encode a fusion protein, for example, to encode a chimeric potassium channel molecule as described herein for expression in hererologous host cells for screening molecules for an ability to modulate hKv9.3 pharmacological activity, i.e., via binding, association or otherwise.

Chimeric constructs may also be used to express a ‘bait’, according to methods well known using a yeast two-hybrid system, to identify accessory native peptides that may be associated with the novel subunit biomolecule described herein. Fields, S., et al., Trends Genet., 10:286 (1994); Allen, J. B., et al., TIBS, 20:511 (1995). A yeast two-hybrid system has been described wherein protein:protein interactions can be detected using a yeast-based genetic assay via reconstitution of transcriptional activators. Fields, S., Song, O., Nature 340:245 (1989). The two-hybrid system used the ability of a pair of interacting proteins to bring a transcription activation domain into close proximity with a DNA-binding site that regulates the expression of an adjacent reporter gene. Commercially available systems such as the CLONTECH, Matchmaker™ systems and protocols may be used with the present invention. CLONTECH, Palo Alto, Calif. See also, Mendelsohn, A. R., Brent, R., Curr. Op. Biotech., 5:482 (1994); Phizicky. E. M., Fields, S., Microbiological Rev., 59(1):94 (1995); Yang, M., et al., Nucleic Acids Res., 23(7): 1152 (1995); Fields, S., Sternglanz, R., TIG, 10(8):286 (1994); and U.S. Pat. Nos. 5,283,173, System to Detect Protein-Protein Interactions, and 5,468,614, which are incorporated herein by reference.

Modified screening systems, for instance, can be practiced either with a positive readout or with a negative readout such as that in the recently developed versions of “Reverse Y2H” approach. See, e.g., Vidal M, Braun P, Chen E, Boeke J D, Harlow E (1996) Genetic characterization of a mammalian protein-protein interaction domain by using a yeast reverse two-hybrid system, Proc Natl Acad Sci USA 17;93(19):10321-10326; Vidal M, Brachmann R K, Fattaey A, Harlow E, Boeke J D (1996) Reverse two-hybrid and one-hybrid systems to detect dissociation of protein-protein and DNA-protein interactions. Proc Natl Acad Sci U S A 17;93(19):10315-10320; White MA (1996) The yeast two-hybrid system: forward and reverse, Proc Natl Acad Sci USA 17;93(19):10001-10003; Leanna C A, Hannink M (1996), The reverse two-hybrid system: a genetic scheme for selection against specific protein/protein interactions, Nucleic Acids Res 1;24(17):3341-3347.

Antibodies

Monospecific antibodies to the biomolecule of the present invention are purified from mammalian antisera containing antibodies reactive against the polypeptide or are prepared as monoclonal antibodies reactive with a human voltage-gated potassium channel polypeptide using the technique of Kohler and Milstein, Nature, 256:495 (1975). Mono-specific antibody as used herein is defined as a single antibody species or multiple antibody species with homogenous binding characteristics for the novel human voltage-gated potassium channel. Homogenous binding as used herein refers to the ability of the antibody species to bind to a specific antigen or epitope. Human voltage-gated modulatory subunit specific antibodies are raised by immunizing animals such as mice, rats, guinea pigs, rabbits, goats, horses and the like, with rabbits being preferred, with an appropriate concentration of the human potassium channel either with or without an immune adjuvant.

Preimmune serum is collected prior to the first immunization. Each animal receives between about 0.1 mg and about 1000 mg of potassium channel polypeptide associated with an acceptable immune adjuvant. Such acceptable adjuvants include, but are not limited to, Freund's complete, Freund's incomplete, alum-precipitate, water in oil emulsion containing Corynebacterium parvum and tRNA. The initial immunization consists of a potassium channel polypeptide in, preferably, Freund's complete adjuvant at multiple sites either subcutaneously (SC), intraperitoneally (IP) or both. Each animal is bled at regular intervals, preferably weekly, to determine antibody titer. The animals may or may not receive booster injections following the initial immunization. Those animals receiving booster injections are generally given an equal amount of the antigen in Freund's incomplete adjuvant by the same route. Booster injections are given at about three week intervals until maximal titers are obtained. At about 7 days after each booster immunization or about weekly after a single immunization, the animals are bled, the serum collected, and aliquots are stored at about −20° C.

Monoclonal antibodies (mAb) reactive with the voltage-gated potassium channel polypeptide are prepared by immunizing inbred mice, preferably Balb/c, with a potassium channel polypeptide. The mice are immunized by the IP or SC route with about 0.1 mg to about 10 mg, preferably about 1 mg, of the novel potassium channel polypeptide in about 0.5 ml buffer or saline incorporated in an equal volume of an acceptable adjuvant, as discussed above. Freund's complete adjuvant is preferred. The mice receive an initial immunization on day 0 and are rested for about 3 to about 30 weeks. Immunized mice are given one or more booster immunizations of about 0.1 to about 10 mg of potassium channel polypeptide in a buffer solution such as phosphate buffered saline by the intravenous (IV) route. Lymphocytes, from antibody positive mice, preferably splenic lymphocytes, are obtained by removing spleens from immunized mice by standard procedures known in the art. Hybridoma cells are produced by mixing the splenic lymphocytes with an appropriate fusion partner, preferably myeloma cells, under conditions which will allow the formation of stable hybridomas. Fusion partners may include, but are not limited to: mouse myelomas P3/NS1/Ag 4-1; MPC-11; S-194 and Sp 2/0, with Sp 2/0 being preferred. The antibody producing cells and myeloma cells are fused in polyethylene glycol, about 1000 molecular weight, at concentrations from about 30% to about 50%. Fused hybridoma cells are selected by growth in hypoxanthine, thymidine and aminopterin supplemented Dulbecco's Modified Eagles Medium (DMEM) by procedures known in the art. Supernatant fluids are collected from growth positive wells on about days 14, 18, and 21 and are screened for antibody production by an immunoassay such as solid phase immunoradioassay (SPIRA) using the human potassium channel polypeptide as the antigen. The culture fluids are also tested in the Ouchterlony precipitation assay to determine the isotype of the mAb. Hybridoma cells from antibody positive wells are cloned by a technique such as the soft agar technique of MacPherson, Soft Agar Techniques, in Tissue Culture Methods and Applications, Kruse and Paterson, Eds., Academic Press, 1973.

Monoclonal antibodies are produced in vivo by injection of pristane primed Balb/c mice, approximately 0.5 ml per mouse, with about 2×10⁶ to about 6×10⁶ hybridoma cells about 4 days after priming. Ascites fluid is collected at approximately 8-12 days after cell transfer and the monoclonal antibodies are purified by techniques known in the art.

In vitro production of the anti-human potassium channel polypeptide mAb is carried out by growing the hydridoma in DMEM containing about 2% fetal calf serum to obtain sufficient quantities of the specific mAb. The mAb are purified by techniques known in the art.

Diagnostic Assays

Antibody titers of ascites or hybridoma culture fluids are determined by various serological or immunological assays which include, but are not limited to, precipitation, passive agglutination, enzyme-linked immunosorbent antibody (ELISA) technique and radioimmunoassay (RIA) techniques. Similar diagnostic assays are used to detect the presence of the novel potassium channel modulatory biomolecule in body fluids or tissue and cell extracts.

Diagnostic assays using the human potassium channel subunit specific antibodies are useful for the diagnosis of conditions, disorders or diseases characterized by abnormal expression of the potassium channel or expression of genes associated with abnormal cell growth or abnormal physiology. Diagnostic assays for the modulatory biomolecule of this invention include methods utilizing the antibody and a label to detect the human potassium channel polypeptide in human body fluids, cells, tissues or sections or extracts of such tissues. The polypeptides and antibodies of the present invention may be used with or without modification. Frequently, the polypeptides and antibodies will be labeled by joining them, either covalently or noncovalently, with a reporter molecule, a myriad of which are well-known to those skilled in the art.

A variety of protocols for measuring the potassium channel polypeptide, using either polyclonal or monoclonal antibodies specific for the respective protein are known in the art. Examples include enzyme-linked immunosorbent assay (ELISA), radioimmunoassay (RIA) and fluorescent activated cell sorting (FACS). A two-site, monoclonal-based immunoassay utilizing monoclonal antibodies reactive to two non-interfering epitopes on the human voltage-gated potassium channel polypeptide is preferred, but a competitive binding assay may be employed. These assays are described, among other places, in Maddox, D. E. et al., J. Exp. Med. 158:1211 (1983); Sites, D. P., et al., Basic and Clinical Immunology, Ch.22, 4th Ed., Lange Medical Publications, Los Altos, Calif. (1982); U.S. Pat. No. 3,654,090, No. 3,850,752; and No. 4,016,043.

In order to provide a basis for the diagnosis of disease, normal or standard values for the human potassium channel polypeptide expression must be established. This is accomplished by combining body fluids or cell extracts taken from normal subjects, either animal or human, with antibody to the human potassium channel biomolecule under conditions suitable for complex formation which are well known in the art. The amount of standard complex formation may be quantified by comparing it with a dilution series of positive controls where a known amount of antibody is combined with known concentrations of purified potassium channel polypeptide. Then, standard values obtained from normal samples may be compared with values obtained from samples from subjects potentially affected by a disorder or disease related to the channel biomolecule expression. Deviation between standard and subject values establishes the presence of the disease state.

Kits containing potassium channel nucleic acid, antibodies to a channel polpeptide, or protein may be prepared. Such kits are used to detect heterologous nucleic acid which hybridizes to potassium channel nucleic acid, or to detect the presence of protein or peptide fragments in a sample. Such characterization is useful for a variety of purposes including, but not limited to, forensic analyses and epidemiological studies.

The DNA molecules, RNA molecules, recombinant protein and antibodies of the present invention may be used to screen and measure levels of the potassium channel subunit DNA, RNA or protein. The recombinant proteins, DNA molecules, RNA molecules and antibodies lend themselves to the formulation of kits suitable for the detection and typing of the novel human potassium channel biomolecule. Such a kit would comprise a compartmentalized carrier suitable to hold in close confinement at least one container. The carrier would further comprise reagents such as recombinant potassium channel or anti-potassium channel antibodies suitable for detecting the novel potassium channel biomolecule. The carrier may also contain a means for detection such as labeled antigen or enzyme substrates or the like.

Polynucleotide sequences which encode the hKv9.3 subunit may be used for the diagnosis of conditions or diseases with which the expression of the novel modulatory biomolecule is associated. For example, polynucleotide sequences encoding the subunit may be used in hybridization or PCR assays of fluids or tissues from biopsies to detect expression of the biomolecule. The form of such qualitative or quantitative methods may include Southern or Northern analysis, dot blot or other membrane-based technologies; PCR technologies; dip stick, pin, chip and ELISA technologies. All of these techniques are well known in the art and are the basis of many commercially available diagnostic kits. Such assays may also be used to evaluate the efficacy of a particular therapeutic treatment regime in animal studies, in clinical trials, or in monitoring the treatment of an individual patient. Once disease is established, a therapeutic agent is administered and a treatment profile is generated. Such assays may be repeated on a regular basis to evaluate whether the values in the profile progress toward or return to the normal or standard pattern. Successive treatment profiles may be used to show the efficacy of treatment over a period of several days or several months.

Polynucleotide sequences which encode the hKv9.3 subunit may also be employed in analyses to map chromosomal locations, e.g., screening for functional association with disease markers. Moreover the sequences described herein are contemplated for use to identify human sequence polymorphisms and possible association with disease as well as analyses to select optimal sequence from among possible polymorphic sequences for the design of compounds to modulate the biological activity and therefore regulate physiological disorders, and pulmonary disorders in vivo. Furthermore the sequences are contemplated as screening tools for use in the identification of appropriate human subjects and patients for therapeutic clinical trials.

Purification Via Affinity Columns

It is readily apparent to those skilled in the art that methods for producing antibodies may be utilized to produce antibodies specific for potassium channel polypeptide fragments, or the full-length nascent human subunit polypeptide. Specifically, it is readily apparent to those skilled in the art that antibodies may be generated which are specific for the fully functional biomolecule or fragments thereof.

Potassium channel subunit antibody affinity columns are made by adding the antibodies to Affigel-10 (Biorad), a gel support which is activated with N hydroxysuccinimide esters such that the antibodies form covalent linkages with the agarose gel bead support. The antibodies are then coupled to the gel via amide bonds with the spacer arm. The remaining activated esters are then quenched with 1M ethanolamine HCl (pH 8). The column is washed with water followed by 0.23M glycine HCl (pH 2.6) to remove any non-conjugated antibody or extraneous protein. The column is then equilibrated in phosphate buffered saline (pH 7.3) with appropriate detergent and the cell culture supernatants or cell extracts, for example, containing human potassium channel polypeptide made using appropriate membrane solubilizing detergents are slowly passed through the column. The column is then washed with phosphate buffered saline/detergent until the optical density falls to background, then the protein is eluted with 0.23M glycine-HCl (pH 2.6)/detergent. The purified subunit polypeptide is then dialyzed against phosphate buffered saline/detergent.

Recombinant potassium channel molecules can be separated from other cellular proteins by use of an immunoaffinity column made with monoclonal or polyclonal antibodies specific for full length nascent human Kv5.1, e.g., SEQ ID NO:3, or polypeptide fragments of the biomolecule.

Human Kv5.1 as described herein may be used to affinity purify biological effectors from native biological materials, e.g. disease tissue. Affinity chromatography techniques are well known to those skilled in the art. The novel polypeptide described herein, e.g., SEQ ID NO:3, or an effective fragment thereof, is fixed to a solid matrix, e.g. CNBr activated Sepharose according to the protocol of the supplier (Pharmacia, Piscataway, N.J.), and a homogenized/buffered cellular solution containing a potential molecule of interest is passed through the column. After washing, the column retains only the biological effector which is subsequently eluted, e.g., using 0.5M acetic acid or a NaCl gradient.

Antisense Molecules

To enable methods of down-regulating expression of the novel potassium channel of the present invention in mammalian cells, an example antisense expression construct containing the complement DNA sequence to the sequence substantially as depicted in SEQ ID NO:2 can be readily constructed for instance using the pREP 10 vector (Invitrogen Corporation). Transcripts are expected to inhibit translation of the wild-type potassium channel mRNA in cells transfected with this type construct. Antisense transcripts are effective for inhibiting translation of the native gene transcript, and capable of inducing the effects (e.g., regulation of pulmonary disorders) herein described. Translation is most effectively inhibited by blocking the mRNA at a site at or near the initiation codon. Thus, oligonucleotides complementary to the corresponding 5′-terminal region of the potassium channel mRNA transcript are preferred. Secondary or tertiary structure which might interfere with hybridization is minimal in this region. Moreover, sequences that are too distant in the 3′ direction from the initiation site can be less effective in hybridizing the mRNA transcripts because of a “read-through” phenomenon whereby the ribosome appears to unravel the antisense/sense duplex to permit translation of the message. Oligonucleotides which are complementary to and hybridizable with any portion of the novel potassium channel mRNA are contemplated for therapeutic use.

U.S. Pat. No. 5,639,595, Identification of Novel Drugs and Reagents, issued Jun. 17, 1997, wherein methods of identifying oligonucleotide sequences that display in vivo activity are thoroughly described, is herein incorporated by reference. Expression vectors containing random oligonucleotide sequences derived from previously known polynucleotides, e.g., SEQ ID NO: 1, are transformed into cells. The cells are then assayed for a phenotype resulting from the desired activity of the oligonucleotide. Once cells with the desired phenotype have been identified, the sequence of the oligonucleotide having the desired activity can be identified. Identification may be accomplished by recovering the vector or by polymerase chain reaction (PCR) amplification and sequencing the region containing the inserted nucleic acid material.

Nucleotide sequences that are complementary to the novel potassium channel polypeptide encoding polynucleotide sequence can be synthesized for antisense therapy. These antisense molecules may be DNA, stable derivatives of DNA such as phosphorothioates or methylphosphonates, RNA, stable derivatives of RNA such as 2′-O-alkylRNA, or other oligonucleotide mimetics. U.S. Pat. No. 5,652,355, Hybrid Oligonucleotide Phosphorothioates, issued Jul. 29, 1997, and U.S. Pat. No. 5,652,356, Inverted Chimeric and Hybrid Oligonucleotides, issued Jul. 29, 1997, which describe the synthesis and effect of physiologically-stable antisense molecules, are incorporated by reference. Potassium channel subunit antisense molecules may be introduced into cells by microinjection, liposome encapsulation or by expression from vectors harboring the antisense sequence. Antisense therapy may be particularly useful for the treatment of diseases where it is beneficial to modulate the biological activity of the potassium channel described herein.

Gene Therapy

A potassium channel polypeptide described herein may administered to a subject via gene therapy. A polypeptide of the present invention may be delivered to the cells of target organs, e.g., pulmonary tissue, in this manner. Conversely, potassium channel polypeptide antisense gene therapy may be used to modulate the expression of the polypeptide in the same cells of target organs and hence regulate biological activity. The potassium channel polypeptide coding region can be ligated into viral vectors which mediate transfer of the trans-activator polypeptide nucleic acid by infection of recipient host cells. Suitable viral vectors include retrovirus, adenovirus, adeno-associated virus, herpes virus, vaccinia virus, polio virus and the like. See, e.g., U.S. Pat. No. 5,624,820, Episomal Expression Vector for Human Gene Therapy, issued Apr. 29, 1997.

Nucleic acid coding regions of the present invention are incorporated into effective eukaryotic expression vectors, which are directly administered or introduced into somatic cells for gene therapy (a nucleic acid fragment comprising a coding region, preferably mRNA transcripts, may also be administered directly or introduced into somatic cells). See, e.g., U.S. Pat. No. 5,589,466, issued Dec. 31, 1996. Such nucleic acids and vectors may remain episomal or may be incorporated into the host chromosomal DNA as a provirus or portion thereof that includes the gene fusion and appropriate eukaryotic transcription and translation signals, i.e, an effectively positioned RNA polymerase promoter 5′ to the transcriptional start site and ATG translation initiation codon of the gene fusion as well as termination codon(s) and transcript polyadenylation signals effectively positioned 3′ to the coding region.

Alternatively, the voltage-gated modulatory subunit DNA can be transferred into cells for gene therapy by non-viral techniques including receptor-mediated targeted DNA transfer using ligand-DNA conjugates or adenovirus-ligand-DNA conjugates, lipofection membrane fusion or direct microinjection. These procedures and variations thereof are suitable for ex vivo, as well as in vivo human gene therapy according to established methods in this art.

PCR Diagnostics

The nucleic acid sequence, oligonucleotides, fragments, portions or antisense molecules thereof, may be used in diagnostic assays of body fluids or biopsied tissues to detect the expression level of the novel human voltage-gated potassium channel molecule. For example, sequences derived from the cDNA sequence SEQ ID NO:1 or sequences comprised in SEQ ID NO:2 can be used to detect the presence of the mRNA transcripts in a patient or to monitor the modulation of transcripts during treatment.

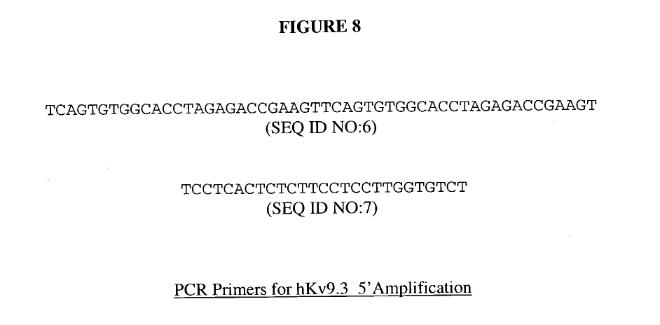

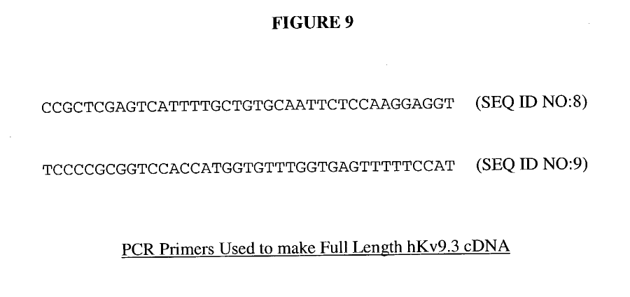

SEQ ID NO:6, SEQ ID NO:7; SEQ ID NO:8, and SEQ ID NO:9, are example primers to be used in PCR diagnostic reactions. FIG. 8 displays PCR primers, SEQ ID NO:6 and SEQ ID NO:7, used for hKv9.3 5′ amplification. FIG. 9 displays PCR primers, SEQ ID NO:8 and SEQ ID NO:9, used to make full length hKv9.3 cDNA.

One method for amplification of target nucleic acids, or for later analysis by hybridization assays, is known as the polymerase chain reaction (“PCR”) or PCR technique. The PCR technique can be applied to detect sequences of the invention in suspected samples using oligonucleotide primers spaced apart from each other and based on the genetic sequence, e.g., SEQ ID NO:1, set forth herein. The primers are complementary to opposite strands of a double stranded DNA molecule and are typically separated by from about 50 to 450 nucleotides or more (usually not more than 2000 nucleotides). This method entails preparing the specific oligonucleotide primers followed by repeated cycles of target DNA denaturation, primer binding, and extension with a DNA polymerase to obtain DNA fragments of the expected length based on the primer spacing. One example embodiment of the present invention is a diagnostic composition for the identification of a polynucleotide sequence comprising the sequence substantially as depicted in SEQ ID NO:2 comprising the PCR primers substantially as depicted in EXAMPLE II. See also, EXAMPLE III. The degree of amplification of a target sequence is controlled by the number of cycles that are performed and is theoretically calculated by the simple formula 2n where n is the number of cycles. See, e.g., Perkin Elmer, PCR Bibliography, Roche Molecular Systems, Branchburg, New Jersey; CLONTECH products, Palo Alto, Calif.; U.S. Pat. No. 5,629,158, Solid Phase Diagnosis of Medical Conditions, issued May 13, 1997.

Compositions

Pharmaceutically useful compositions comprising sequences pertaing to the novel potassium channel polypeptide, DNA, RNA, antisense sequences, or the human polypeptide itself, or variants and analogs which have biological activity or otherwise compounds which modulate cell physiology identified by methods described herein, may be formulated according to known methods such as by the admixture of a pharmaceutically acceptable carrier. Examples of such carriers and methods of formulation may be found in Remington's Pharmaceutical Sciences (Maack Publishing Co, Easton, Pa.). To form a pharmaceutically acceptable composition suitable for effective administration, such compositions will contain an effective amount of the protein, DNA, RNA, or compound modulator.

Therapeutic or diagnostic compositions of the invention are administered to an individual in amounts sufficient to treat or diagnose human physiological disorders or pulmonary disorders. The effective amount may vary according to a variety of factors such as the individual's condition, weight, sex and age. Other factors include the mode of administration.

The term “chemical derivative” describes a molecule that contains additional chemical moieties which are not normally a part of the base molecule. Such moieties may improve the solubility, half-life, absorption, etc. of the base molecule. Alternatively the moieties may attenuate undesirable side effects of the base molecule or decrease the toxicity of the base molecule. Examples of such moieties are described in a variety of texts, such as Remington's Pharmaceutical Sciences.

Pharmaceutical compositions suitable for use in the present invention include compositions wherein the active ingredients are contained in an effective amount to achieve the intended purpose. The determination of an effective dose is well within the capability of those skilled in the art. The therapeutically effective dose can be estimated initially either in cell culture assays, eg, of neoplastic cells, or in animal models, usually mice, rabbits, dogs, or pigs. The animal model is also used to achieve a desirable concentration range and route of administration. Such information can then be used to determine useful doses and routes for administration in humans. A therapeutically effective dose refers to that amount of protein or its antibodies, antagonists, or inhibitors which ameliorate the symptoms or condition. The exact dosage is chosen by the individual physician in view of the patient to be treated.

Compounds identified according to the methods disclosed herein may be used alone at appropriate dosages defined by routine testing in order to obtain optimal modulation of a potassium channel biological activity and/or physiological condition, or its activity while minimizing any potential toxicity. In addition, co-administration or sequential administration of other agents may be desirable.

The pharmaceutical compositions may be provided to the individual by a variety of routes such as subcutaneous, topical, oral and intramuscular. Administration of pharmaceutical compositions is accomplished orally or parenterally. Methods of parenteral delivery include topical, intra-arterial (directly to the tissue), intramuscular, subcutaneous, intramedullary, intrathecal, intraventricular, intravenous, intraperitoneal, or intranasal administration. The present invention also has the objective of providing suitable topical, oral, systemic and parenteral pharmaceutical formulations for use in the novel methods of treatment of the present invention. The compositions containing compounds identified according to this invention as the active ingredient for use in the modulation of physiological conditions can be administered in a wide variety of therapeutic dosage forms in conventional vehicles for administration. For example, the compounds can be administered in such oral dosage forms as tablets, capsules (each including timed release and sustained release formulations), pills, powders, granules, elixirs, tinctures, solutions, suspensions, syrups and emulsions, or by injection. Likewise, they may also be administered in intravenous (both bolus and infusion), intraperitoneal, subcutaneous, topical with or without occlusion, or intramuscular form, all using forms well known to those of ordinary skill in the pharmaceutical arts. An effective but non-toxic amount of the compound desired can be employed as a subunit or potassium channel modulating agent.

The daily dosage of the products may be varied over a wide range from 0.01 to 1,000 mg per adult human/per day. For oral administration, the compositions are preferably provided in the form of scored or unscored tablets containing 0.01, 0.05, 0.1, 0.5, 1.0, 2.5, 5.0, 10.0, 15.0, 25.0, and 50.0 milligrams of the active ingredient for the symptomatic adjustment of the dosage to the patient to be treated. An effective amount of the drug is ordinarily supplied at a dosage level of from about 0.0001 mg/kg to about 100 mg/kg of body weight per day. The range is more particularly from about 0.001 mg/kg to 10 mg/kg of body weight per day. Even more particularly, the range varies from about 0.05 to about 1 mg/kg. Of course the dosage level will vary depending upon the potency of the particular compound. Certain compounds will be more potent than others. In addition, the dosage level will vary depending upon the bioavailability of the compound. The more bioavailable and potent the compound, the less compound will need to be administered through any delivery route, including but not limited to oral delivery. The dosages of the modulators desribed herein are adjusted when combined to achieve desired effects. On the other hand, dosages of these various agents may be independently optimized and combined to achieve a synergistic result wherein the pathology is reduced more than it would be if either agent were used alone. Those skilled in the art will employ different formulations for nucleotides than for proteins or their inhibitors. Similarly, delivery of polynucleotides or polypeptides will be specific to particular cells and conditions.

### EXAMPLES

### EXAMPLE I

Identification

A novel full-length potassium channel cDNA sequence belonging to the voltage-gated family was isolated from a human brain cDNA library. The novel cDNA is expressed in several tumor tissues. High-level expression of the novel gene either as a homomultimer or as a heterotetramer with other K channels, allows rapid screening of small molecules for use as therapeutics in disease conditions including pulmonary disorders.

The initial lead into this sequence was obtained by searching a proprietary database for novel potassium channel sequences using the pore motif of voltage-gated potassium channels. The pore sequence of hKv1.1 (PDAFWWAVVSMTTVGYGDMY) (SEQ ID NO:5), a Shaker-related K channel (Chandy and Gutman, Handbook of Receptors and Channels, Ligand- and Voltage-gated Ion Channels, edited by R. Alan North, CRC Press, Inc., 1995, Chapter 1) was used as a query sequence in a tblastn search (Altschul, SF et al.,. J. Mol. Biol. 215:403-10, 1990). A sequence was identified from colon tumor tissue (annotated as hKv1.3) which was subsequently used as a query sequence in a blastn search to identify an overlapping clone from small intestine tumor tissue. The two clones were sequenced in-house (ABI PRISM™ Dye Terminator Cycle sequencing on ABI PRISM™377 automated sequencer). An independent search for novel potassium channels using the voltage sensor motif RIMRILRILKLAR (SEQ ID NO:11) identified a new clone which was the 5′ most clone. An electronic Northerm was performed using the new clone (Table II). Five out of the eight tissues were tumor tissue.

TABLE II
ELECTRONIC NORTHERN

| Library | Lib Description | Abun | Pct Abun |
|---|---|---|---|
| SININOT04 | small intestine, ileum, Crohn's, 26 M | 2 | 0.0481 |
| COLNNOT08 | colon, 60 M, match to COLNTUT16 | 1 | 0.0426 |
| | (tumor) | | |
| COLNTUT03 | colon tumor, 62 M, match to | 1 | 0.0196 |
| | COLNNOT16 | | |
| MUSCNOT07 | muscle, forearm, 38 F | 1 | 0.0154 |
| CONUTUT01 | mesentery tumor, sigmoid, 61 F | 1 | 0.0130 |
| BRSTTUT01 | breast tumor, 55 F, match to | 1 | 0.0095 |
| | BRSTNOT0 | | |
| CARDFEM01 | heart, fetal, NORM, WM | 1 | 0.0082 |
| BRSTNOT05 | breast, 58 F, match to BRSTTUT03 | 1 | 0.0075 |
| | (tumor) | | |

### EXAMPLE II

5′ Race

5′ RACE techniques (rapid amplification of cDNA ends) were used to identified the start codon of the channel cDNA. Two primers were designed according to the sequence of contig (positions 1423-1398 of SEQ ID NO: 1) (SEQ ID NO:6) and (positions 1294-1268 of SEQ ID NO:1) (SEQ ID NO:7). High-fidelity Advantage KlenTaq polymerase mix from CLONTECH was used in the RACE-PCR amplification. Human brain cDNA (CLONTECH) was used as templates for RACE-PCR amplification. The primers were paired with primers AP1 and AP2, respectively, which were supplied with the Advantage KlenTaq polymerase. The first round PCR (primed with SEQ ID NO:6 and API) was carried out for a total of 30 cycles (5 cycles of 94° C. for 0.5 min, 72° C. for 4 min, 5 cycles of 94° C. for 0.5 min, 70° C. for 4 min, 10 cycles of 94° C. for 0.5 min, and 68° C. for 4 min, 10 cycles of 94° C. for 0.5 min, 66° C. for 4 min). The second round PCR (primed with SEQ ID NO:7 and AP2) was carried our 35 cycles of 94° C. for 0.5 min, 72-68° C. for 3 min (first 10 cycles with a decrement of 0.4° C./cycle from 72° C. to 68° C. and the final 25 cycles with 68° C.). The second 5′ RACE products were cloned into pCRII (INVITROGEN) for sequencing. Four clones sequenced were all identified as Kv channel. By assembling the sequences of the 5′ RACE clones and clones, an ORF encoding a novel Kv channel was identified. The sequence at positions −5 to 4 of the ORF is 100% identical to the Kozak consensus sequence. Five in-frame stop codons are present at positions-51, -93, -117, -120 and -258 for this ORF. Thus, the initiation codon is assigned to the first methionine of this ORF.

### EXAMPLE III

Full-Length Cloning

PCR techniques was used to clone the full-length cDNA sequence (SEQ ID NO:1) for the novel channel. Two primers with restriction digesting sites were designed according to the assembled sequence (XhoI)-CCG CTC GAG TCA TTT TGC TGT GCA ATT CTC CAA GGA GGT (positions 1928-1899 in SEQ ID NO:1) (SEQ ID NO:8) and (SacII)-TCC CCG CGG TCC ACC ATG GTG TTT GGT GAG TTT TTC CAT (positions 50-79 in SEQ ID NO:1) (SEQ ID NO:9) and used to direct the PCR amplification. High-fidelity Advantage KlenTaq polymerase mix from CLONTECH was used in the amplification. Human brain cDNA (CLONTECH) was used as templates. PCR was carried out 30 cycles of 94° C. for 0.5 min, 62° C. for 0.5 min, and 68° C. for 3.5 min. The PCR products were cloned into pKGEM vector by digesting both PCR product and vector with XhoI and SacII. Three clones were submitted for sequencing and the sequencing results confirmed that the full-length cDNA clone encoded a novel human protein for a Kv channel (SEQ ID NO:1). The cDNA sequence (SEQ ID NO:1) and translated protein sequence (SEQ ID NO:3) for the novel hKv9.3 channel was identified on Jul., 18, 1997; at this time there was no report of this gene sequence in any public databases.

### EXAMPLE IV

Baculoviral Overexpression System for Screening Preparation

(Generation of recombinant baculoviral expression vectors and gene expression with the BAC-TO-BAC Expression System, Life Technologies, Gaithersburg, Md. Cat. # 10359-016 BAC to BAC Baculovirus Expression System) SEQ ID NO:2 is cloned into pFASTBAC1, and the recombinant plasmid is transformed into DH10BAC competent cells which contain the bacmid with a mini-attTn7 target site and the helper plasmid. The mini-Tn7 element on the pFASTBAC I plasmid can transpose to the mini-attTn7 target site on the bacmid in the presence of transposition proteins provided by the helper plasmid. Colonies containing recombinant bacmids are identified by disruption of the IacZα gene. High molecular weight mini-prep DNA is prepared from selected E. coli clones containing the recombinant bacmid, and this DNA is then used to transfect insect cells.

Cloning into pFASTBAC1

The donor vector, pFASTBAC1, contains the polyhedrin promoter followed by an extensive MCS which extends from BamH I (4032) to Hind m (4137). The BamH I site is 37 bp downstream from the original ATG of the polyhedrin gene, which has been mutated to ATT. Therefore, to successfully express a foreign protein, the foreign DNA fragment must contain its own ATG followed by an open reading frame (ORF). The foreign DNA fragment must be cloned into pFASTBAC1 in the correct orientation with respect to the polyhedrin promoter (i.e., the 5′ end of the gene must be inserted into the first selected site of the MCS).

Prepare pFASTBAC1 and the foreign DNA fragment by digesting 500 ng to 1 μg DNA with the selected restriction endonuclease(s) under the appropriate conditions. If only one site in the vector is chosen as the cloning site, dephosphorylate the vector under the appropriate conditions. DNA fragments can be purified by agarose gel electrophoresis and the fragments of interest can be recovered from the gel by using a GLASSMAX® cartridge or an equivalent purification. Ligate the prepared vector and insert fragments under the appropriate conditions.

Cloning

For this initial cloning, do not use the DH10BAC cells in the system. DH5α or DH10B competent cells can be used. Plate the transformation mix onto LB agar plates containing 100 μg/ml ampicillin.

For analysis of a directional cloning experiment, 6 colonies are sufficient to screen; 12 or more may need to be analyzed for a nondirectional cloning strategy. Prepare plasmid DNA from overnight cultures using a mini-preparation procedure (e.g., Schecter, A. I., et al., Nature 312:513 (1984) and verify correct insertion of the gene of interest by restriction endonuclease digestion or PCR analysis.

Transformation

1. Prepare Luria Agar plates containing:

50 μg/ml kanamycin,

7 μg/ml gentamycin;

10 μg/ml tetracycline;

300 μg/ml Bluo-gal and

40 μg/ml IPTG.

See additional protocols for formulations (Sections 5.1 and 5.2)

2. Thaw the DH10BAC competent cells on ice.

3. Dispense 100 μl of the cells into 15-ml polypropylene tubes.

4. Add approximately 1 ng recombinant donor plasmid (in 5 μl) and gently mix the DNA into the cells by tapping the side of the tube/

5. Incubate the mixture on ice for 30 min.

6. Heat shock the mixture by transferring to 42° C. water bath for 45 s.

7. Chill the mixture on ice for 2 min.

8. Add 900 μl S.O.C. medium to the mixture.

9. Place the mixture in a shaking incubator at 37° C. with medium agitation for 4 h.

10. Serially dilute the cells, using S.O.C. medium, to 10⁻¹, 10⁻², 10⁻³, (i.e., 100 μl of transposition mix: 900 μl of S.O.C. medium=10⁻¹ dilution, use this to further dilute 10-fold to give 10⁻² dilution, and similarly for 10⁻³ dilution).

11. Place 100 μl of each dilution on the plates and spread evenly over the surface.

12. Incubate for at least 24 h at 37° C. (Blue colonies may not be visible prior to 24 h).

Isolation of Recombinant DNA

White colonies contain the recombinant bacmid, and therefore, are selected for isolation of recombinant bacmid DNA. Before isolating DNA, candidate colonies are streaked to ensure they are truly white.

1. Select white colonies from a plate with approximately 100 to 200 colonies. NOTE: This number facilitates differentiation between blue and white colonies.

2. Pick—10 white candidates and streak to fresh plates to verify the phenotype. Incubate overnight at 37° C.

3. From a single colony confirmed as having a white phenotype on plates containing Bluo-gal and IPTG, set up a liquid culture for isolation of recombinant bacmid DNA.

The following protocol is specifically developed for isolating large plasmids (>100 kb), and is adapted for isolating bacmid DNA.

1. Using a sterile toothpick, inoculate a single, isolated bacterial colony into 2 ml LB medium supplemented with 50 μg/ml kanamycin, 7 μg/ml gentamicin, and 10 μg/ml tetracycline. A 15-ml snap-cap polypropylene tube is suitable. Grow at 37° C. to stationary phase (up to 16 h) shaking at 250 to 300 rpm.

2. Transfer 1 ml of culture to a 1.5-ml microcentrifuge tube and centrifuge at 14,000×g for 1 min.

3. Remove the supernatant by vacuum aspiration and resuspend (by gently vortexing, or pipetting up and down, if necessary) each pellet in 0.3 ml of Solution I [15 mM Tris-HCl (pH 8.0), 10 mM EDTA, 100 μg/ml Rnase A]. Add 0.3 ml of Solution II (0.2 N NaOH, 1% SDS) and gently mix. Incubate at room temperature for 5 min. Note: The appearance of the suspension should change from very turbid to almost translucent.

4. Slowly add 0.3 ml of 3 M potassium acetate (pH 5.5), mixing gently during addition. A thick white precipitate of protein and genomic E. coli DNA will form. Place the sample on ice for 5 to 10 min.

5. Centrifuge for 10 min. at 14,000×g. During the centrifugation, label another microcentrifuge tube and add 0.8 ml absolute isopropanol to it.

6. Gently transfer the supernatant to the tube containing isopropanol. Avoid any white precipitate material. Mix by gently inverting tube a few times and place on ice for 5 to 10 min. At this stage, the sample can be stored at −20° C. overnight.

7. Centrifuge the sample for 15 min. at 14,000×g at room temperature.

8. Remove the supernatant and add 0.5 ml 70% ethanol to each tube. Invert the tube several times to wash the pellet. Centrifuge for 5 min. at 14,000×g at room temperature. (Optional: repeat step 8.)

9. Remove as much of the supernatant as possible. Note: The pellet may become dislodged from the bottom of the tube, so it is better to carefully aspirate the supernatant than to pour it.

10. Air dry the pellet briefly, 5 to 10 min., at room temperature and dissolve the DNA in 40 μl TE. Allow the solution to sit in the tube with occasional gentle tapping of the bottom of the tube. The DNA is generally ready for use within 10 min., as long as the pellets are not overdried.

11. Store the DNA at −20° C. However, avoid repeated freeze/thaw cycles to avoid a drastic reduction in transfection efficiency.

Preparations of bacmid DNA may be analyzed by agarose gel electrophoresis to confirm the presence of high molecular weight DNA.

Transfection of Sf9 Cells with Recombinant Bacmid DNA

1. Seed 9×10⁵ cells per 35-mm well (of a 6-well plate) in 2 ml of Sf-900 II SFM containing penicillin/streptomycin at 0.5×final concentration (50 units/ml penicillin, 50 up/ml streptomycin).

2. Allow cells to attach at 27° C. for at least 1 h.

3. Prepare the following solutions in 12-×75-mm sterile tubes:

Solution A; For each transfection, dilute ˜5 up of mini-prep bacmid DNA into 100 μl Sf-900 II SFM without antibiotics.

Solution B; For each transfection, dilute ˜6 ul CELLFECTIN Reagent into 100 μl Sf-900 II SFM without antibiotics.

4. Combine the two solutions, mix gently, and incubate for 15 to 45 min. at room temperature.

5. Wash the cells once with 2 ml of Sf-900 II SFM without antibiotics.

6. For each transfection, add 0.8 ml of Sf-900 II SFM to each tube containing the lipid-DNA complexes. Mix gently. Aspirate wash media from cells and overlay 1 ml of the diluted lipid-DNA complexes onto the cells.

7. Incubate cells for 5 h in a 27° C. incubator.

8. Remove the transfection mixtures and add 2 ml of Sf-900 II SFM containing antibiotics. Incubate cells in a 27° C. incubator for 48 h.

9. Assay cells for protein activity 48 to 72 h after the start of transfection; harvest virus at 72 h.

Harvest/Storage of Recombinant Baculovirus

1. When harvesting virus from the transfection, transfer the supernatant (2 ml) to a sterile, capped tube. Clarify by centrifugation for 5 min. at 500×g and transfer the virus-containing supernant to a fresh tube.

2. From the initial transfection, viral titers of 2×10⁷ to 4×10⁷ pfu/ml can be expected.

3. Store the virus at 4° C., protected from light. For long term storage of virus, the addition of fetal bovine serum (FBS) to a final concentration of at least 2% FBS is recommended. Storage of an aliquot of the viral stock at −70° C. is also recommended.

4. For determining the viral titer, a plaque assay can be performed. See Section 5.12, Viral Plaque Assay, for plaquing procedures.

5. For amplifying viral stocks, infect a suspension or monolayer culture at a Multiplicity of Infection (MOI) of 0.01 to 0.1. Use the following formula:

$$Innoculum required ( ml ) \text{:} \frac{desired MOI ( pfu \text{/} ml ) \times ( total number of cells )}{titer of virual innoculum ( pfu \text{/} ml )}$$

For example, infect a 50-ml culture at 2×10⁶ cells/ml with 0.5 ml of a viral stock that is 2×107 pfu/ml, for an MOI of 0.1.

Harvest virus at 48 h post-infection. This will result in approximately 100-fold amplification of the virus.

Infection of Insect Cells with Recombinant Baculovirus Particles

Optimal infection conditions for insect cells can vary. A starting point for infection is an MOI of 5 to 10. For more information, please refer to reference 2. It is recommended that several experiments be performed for each protein to be expressed.

MOI optimization: Infect a population of cells at varying MOIs (e.g., 1, 2, 5, 10) and assay protein expression upon harvesting the cells (or media, if the protein is secreted).

Time course: Infect cells at a constant MOI. Harvest cells (or media) at the following time intervals: 24 h, 48 h, 72 h, and 96 h. Assay for expression.

Preparing the Cells for Transfection

1. Twenty-four hours before transfection, inoculate a 60-mm tissue culture dish with 4×10⁵-11×10⁵ exponentially growing cells. The cells should be 60-80% confluent at the time of transfection.

2. Grow the cells overnight in 5 ml of the appropriate culture medium.

Preparing the Complex Formation Solution

1. Transfer 900 μl of sterile serum-free, antibiotic-free DMEM (DMEM-SA) to a polystyrene tube.

2. Add 35-100 μl of LipoTAXI transfection reagent. Tap the side of the tube to mix.

3. Add 7 μg of the control plasmid to the control reaction and 5-10 μg of the experimental DNA to the experimental reaction. For stable transfections, prepare a negative control.

4. Mix gently (do not vortex) and incubate for 15-30 minutes at room temperature.

Adding the Activated Solution

1. Remove the standard culture medium from the tissue culture dish by aspiration.

2. Add 1.5 ml of DMEM (serum optional) to the transfection mixture in the polystyrene tube and then transfer this entire mixture (˜2.5 ml) dropwise to the tissue culture dish while swirling the dish.

3. Incubate for 4-6 hours using standard growth conditions (i.e., 37° C. and 5% CO₂ in a humidified incubator).

4. Add 2.5 ml of DMEM containing serum (DMEM+S) at twice the normal serum concentration to the tissue culture dish and incubate overnight.

5. Replace the medium with 5.0 ml of fresh, complete medium.

6. Incubate the cells for 24-72 hours depending on the cell type, reporter system, and promoter activity or proceed to Performing a Stable Transfection.

Performing a Stable Transfection

1. Split the cells from step 6 of Adding the Activated Solution to the desired ratio (at least 1:10) after the 24-hour incubation and then incubate overnight.

2. Apply selection antibiotics dropwise to the tissue culture dish, swirling the dish between drops, at a concentration appropriate to the cell line.

3. Replace the medium and apply fresh selection antibiotics every 4-7 days (approximately two times per week).

4. Stable colonies form within 1-2 weeks. Cells from the negative DNA control dish die off.

Transfecting Suspension Cells with the LipoTAXI Mammalian Transfection Kit

Preparing the Cells for Transfection

1. Seed 4×10-10×10⁶ cells per 60-mm dish in 700 μl of DMEM-SA.

Note The optimal ratio of LipoTAXI transfection reagent to DNA must be determined for each plasmid and cell line.

Preparing the Complex Formation Solution

1. Transfer 900 μl of DMEM-SA to a polystyrene tube.

2. Add 35-100 μl of LipoTAXI transfection reagent. Tap the side of the tube to mix.

3. Add 10 μg of the control plasmid to the control reaction and 7-15 μg of the experimental DNA to the experimental reaction. For stable transfections, prepare a negative control.

4. Mix gently (do not vortex) and incubate for 15-30 minutes at room temperature.

Adding the Activated Solution

1. Add 800 μl of DMEM (serum optional) to the transfection mixture in the polystyrene tube and then transfer this entire mixture (˜1.8 ml) dropwise to the tissue culture dish while swirling the dish.

2. Incubate for 4-6 hours using standard growth conditions (i.e., 37° C. and 5% CO₂ in a humidified incubator).

3. Add 3 ml of fresh, complete DMEM to the tissue culture dish.

4. Incubate the cells for 24-72 hours depending on the cell type, reporter system, and promoter activity or proceed to step 10 if performing a stable transfection.

Performing a Stable Transfection

1. After 48 hours, seed a fresh tissue culture dish to be used for selection at no greater than one-third the density of the transfected cells from step 9.

2. Apply selection antibiotics dropwise to the tissue culture dish, swirling the dish between drops, at a concentration appropriate to the cell line.

3. Replace the medium and apply fresh selection antibiotics every 4-7 days (approximately two times per week).

4. Stable colonies form within 1-2 weeks. Cells from the negative DNA control dish die off.

Harvesting the Transfected Suspended Cells

1. Transfer the transfected cells into 15-ml centrifuge tubes and spin for 5 minutes at 200×g to pellet the cells.

2. Remove and discard the supernatant, add 1 ml of PBS to the cell pellet, and transfer the resulting cell suspension to a 1.7-ml microcentrifuge tube.

3. Spin the tube in a microcentrifuge at 200×g for 5 minutes to pellet the cells.

4. Remove and discard the supernatant, add 1 ml of lysis buffer to the cell pellet, and place at −20° C. until frozen.

5. Thaw the cell extract and spin the tube in a microcentrifuge at 12,000×g for 5 minutes to pellet the cell debris.

6. Transfer the supernatant to a new microcentrifuge tube and store at −20° C. or proceed to B-Galactosidase Assay.

### EXAMPLE V

CaPO₄ Transfection Protocol

The following protocol is optimized for 100-mm culture dishes and all volumes are for single transfection.

1. Inoculate 100-mm culture dishes with exponentially growing cells at 5×10⁵ cells per dish no more than 24 hours before the transfection. These cells should be grown overnight in 10 ml of the appropriate medium to a confluency of approximately 10-20%. The RPMI series of media cannot be used for CaPO₄ transfection. The excess positive charge in this media will cause a dense precipitate to form.

Preparation of the DNA Precipitate Solution (per 100-mm culture dish)

NOTE: The optimal amount of DNA will vary depending on the cell type being transfected. For the control plasmid p WLneo (Life Technologies, Gaithersburg, Md.), use 10 μg/dish, (10-30 μg of circular DNA/dish is recommended in most cases).

2. Dilute desired amount of DNA with distilled, deionized water to 450 μl.

3. Allow mixture to incubate 10-20 minutes at room temperature.

4. Gently mix the solution to ensure adequate suspension. Add this to the culture dropwise and swirl plate gently to distribute evenly.

5. Incubate cells for 12-24 hours. Depending upon cell type, optimal incubation time may vary. A fine precipitate will be seen after incubation.

NOTES: If spare incubator space is available, transfection efficiency can be improved 2-3 times by using lower CO₂ concentrations (2-4% CO₂).

Return to normal CO₂ concentrations after precipitation removal in the following step (step 6).

6. After 12-24 hours incubation, remove medium and rinse culture twice using PBS (without Ca or Mg), or medium without serum. Apply fresh, complete medium (serum-containing), and allow the cells to incubate 24 hours under a CO₂ concentration optimal for the cell line.

7. Split cells at desired rations (at least 1:10) and incubate an additional 24 hours before applying selection of stable transfectants.

### EXAMPLE VI

DEAE-Dextran Transfection Protocol

The following protocol has been optimized for 100-mm culture dishes and all volumes are for a single transfection. For the control plasmid pSV2 Cat (Life Technologies, Gaithersburg, Md.) use 2 μg/100 mm dish.

DEAE-Dextran Transfection (per 100-mm Culture Dish)

1. Exponentially growing cells should be seeded at a density which will yield—100% confluency within 72 hours. Transfection should be performed between 6-24 hours after seeding.

2. Mix DNA (1-2 μg/100-mm dish) with phosphate buffered saline (1×PBS) to a final volume of 170 μl.

3. Dilute 85 μl of Solution #3 with 85 μl of 1×PBS.

4. Combine the PBS-DNA mixture with the Solution #3-PBS mixture.

5. Remove media from cultures and rinse twice with PBS.

6. Add the DNA mixture (from step 4) dropwise to the center of the culture and swirl gently to distribute the solution evenly. Incubate for 15 minutes at room temperature (−25° C.) without CO₂.

7. Remove the solution and gently rinse the culture with PBS (cells will be poorly attached at this point).

8. Add 10 ml of fresh, complete media to the culture and allow to grow under optimal culture conditions.

Selection

When using the G418 antibiotic selection method, it is important to remember that no all mammalian cell lines are equally sensitive to G418. The minimal lethal concentration can range from 100 μg/ml to 1 mg/ml. Therefore, the concentration to be used for selection must be determined for each cell line before the experimental can begin.

Since many cell lines have already been subjected to this type of selection, it may be useful to consult the available literature. If no information about the sensitivity of a particular cell line is available, a simple way to determine its sensitivity is to grow cultures in a multiwell late with a range of G418 concentrations between the individual wells. The optimal concentration is the lowest one that kills all of the cells within 10-14 days. (Rapidly dividing cells may be killed more readily since the antibiotic appears to act mainly on dividing cells.)

In some cases, it may be possible to reduce the concentration of G418 after the initial selection and still maintain the selectable marker gene. For example, NIH 3T3 cells are generally selected in 400 μg/ml G418 and the presence of the neor gene can be maintained in 250 μg/ml.

Clone Isolation

After the selection process progresses to the point where colonies are visible, it is important to isolate the colonies in a manner that obtains the maximum number of cells (increasing the likelihood the clone will survive), and to minimize the changes of contaminating one colony with cells from another. To help achieve both these goals, it is recommend use cloning rings when isolating colonies. The rings may be made of any autoclavable material, and have an internal diameter of 5-10 mm. Sterilize them along with a pair of medium forceps and some form of adhesive (vacuum grease works well).

Before isolating the colonies, prepare 24-well microtiter dishes to receive the new clones. The volume used for the new clones should be kept to a minimum, since many types of cells appear to require the support of other cells of the same type in order to grow in culture. This support may involve some kind of soluble growth factor which may become ineffective if diluted too much. In cases where cell growth is initially very sparse (<5% of the dish surface), it is often helpful to add growth factors at higher concentrations than usual.

NOTE: For example, if a cell type grows wells in media supplemented with 10% fetal calf serum under normal culture conditions, it may require 20% fetal calf serum to grow well when the cells are seeded at a very low density. It may also be useful to remove media from a more densely populated culture and, after sterile filtering, use it to supplement the growth of medium of the sparsely populated cultures.

To isolate single colonies using cloning rings, the culture containing the colonies should be washed twice in PBS, then all fluid should be removed from the plate. The rings should be handled only with sterile forceps.

1. Dip the rings in the sterile adhesive and lightly dab them against a dry, sterile surface to spread the adhesive evenly around the outer edges.

2. Apply the rings around the colonies to be harvested (work quickly to prevent drying out).

3. Add 4-5 drops of trypsin to each ring with a sterile, cotton-plugged Pasteur pipet.

4. Wait ˜30 seconds per colony, then break up the colony by pipetting the trypsin up and down 2-3 times.

5. Transfer the trypsinized cells to a 24-well plate.

6. Remove some of the media from the 24-well late, rinse the inside of the ring to remove any residual cells and transfer to the dish.

7. After ˜2 hours, examine the plate with a microscope to determine if the cells have attached. If they have attached, replace the media in the 24-well plate with fresh, complete media in order to remove any remaining trypsin.

During the early stages of new clone growth, it is best to change the media as infrequently as possible (about once a week for very sparsely populated cultures), which helps maintain any soluble factors. It is best to add exogenous growth factors to the media, instead of replacing the old media with media containing fresh growth factors.

Once the small cultures have grown to confluency, they may be passed to larger culture vessels (usually 6-well microtiter plates first) and treated the same way as other cells of the same type (the original selection should be maintained throughout the life of the new clone).

### EXAMPLE VII

Membrane Preparation of Transfected HEK Cells for Screening Compounds

Membranes are prepared when the HEK cells in each T-150 (or T-175 or T-225) flask are confluent.

The preparation of membranes from each flask of cells is as follows:

1. Pour off the cell culture media and save (since it always contains some cells that have just been dislodged from handling the flask).

2. Rinse the confluent layer of cells with about 8 mls of phosphate buffered saline (PBS), 37° C. This removes residual fetal calf serum which can inhibit the trypsin used in step 3. Save the PBS from the rinse, too, to capture any other cells that are dislodged.

3. Add about 5 ml of trypsin-EDTA, 37° C. Slowly rock the flask back and forth to let the trypsin wash over the whole layer of cells. In about 30 sec, some of the cells will come loose; tap the flask firmly on the lab bench to dislodge the rest of the cells. Quickly add fresh medium to the flash to stop the trypsin reaction. Add about 10 ml medium for every 5 ml of trypsin. Pipet the trypsin/cells/medium into a centrifuge tube. Rinse the flask with about 5 ml more medium and combine with other tubes.

4. Wash the cells by spinning at 250×g, 8 min. Resuspend the individual cell pellets in a small volume of assay buffer, and combine them into one tube.

5. Homogenize the cells using a Polytron, 25 sec., 13000 RPM

6. LOW SPEED SPIN: 800 RPM (˜100×g) 2 min.; save the supernatant; discard the pellet

7. HIGH SPEED SPIN: transfer the sup into high speed tubes, and spin 20,000 RPM (50,000×g), 10 min. Discard the supernatant; add 1 ml of assay buffer on top of pellet

8. Freeze on dry ice; cap the tubes and store at −80 C.

Dilute the membranes to 50 ug/ml, and use 2.5 ug in each well (50 ul of 50 ug/ml)

### EXAMPLE VIII

Radiolabelled Ligand Competitive Binding in Membranes from Transfected HEK cells 96 Well Microtiter Plate Method; Separation of Bound and Free by Filtration over GF/C Filters

Radiolabelled ligands for use in this example include but are not limited to tetraethylammonium chloride (TEA), 4-aminopyridine (4-AP, as well as 2-AP and 3-AP), 3,4- and 2,3-diaminopyridine, BaCl₂, CsCl, strychnine, phencyclidine, pyridostigmine, 9-aminoacridine, DuP-996 (3,3-bis (4-pyridinylmethyl)-1-phenylindolin-2-one; linopiridine), clofilium, quinidine, aminoquinolines and quinine.

| Assay Buffer: | Wash Buffer: |
|---|---|
| 5 mM NaCl | 200 mM NaCl |
| 5 mM KCl | 20 mM HEPES |
| 10 mM HEPES | pH 8.0 with Tris base |
| 6 mM glucose | |
| bring to pH 8.4 with TRIS base | |
| membranes: | transfected HEK cell membranes |
| | Use 2.5 ug protein per well in the assay |
| misc. | 96 well microtiter plates, Falcon, round |
| | bottom, non tissue culture treated |
| | 96 well Unifilters, GF/C, Packard |
| | Instrument Co. |
| | Microscint-20 scintillation fluor, |
| | Packard |
| | Top Count scintillation counter, Packard |
| | Polyethylenimine, 0.6% for soaking |
| | Unifilters |
| | Radiolabelled ligand, e.g. TEA, 2200 |
| | Ci/mmol |
| | nonspecific binding defined with 10 |
| | nM cold Ligand (e.g., TEA) |
| Assay: | 130 ul assay buffer |
| | 20 ul test compound or control or |
| | nonspecific |
| | 50 ul membranes at 50 ug/ml (2.5 ug |
| | protein added to each well) |
| | 50 ul Radioligand [final] 25 pM |
| | 250 ul total volume |

Assay Method:

Place 130 ul per well in 96 well plate

Add 20 ul of candidate compound (drug), or control solution (more buffer or test compound diluent), or nonspecific (defined with 10 nM cold Ligand)

Add the previously prepared membranes to the assay plate, and pre-incubate membranes, plus buffer, test compounds, etc. for 5 min, 21° C., mixing on a Titer-Tek plate mixer

Add 50 ul Radioligand, [final] 25 pM, and incubate for 20 min. 21° C., mixing on the plate mixer

Separate bound from free Radioligand by filtering over the presoaked GF/C Unifilters; wash twice rapidly using ice cold wash buffer

Let Unifilter plates dry overnight

When dry, add 25 ul Microscint-20 to Unifilters, and count in Top Count (Packard) scintillation counter

### EXAMPLE IX

Radioligand Binding Assays

Radiolabelled ligands for use in this example include but are not limited to tetraethylammonium chloride (TEA), 4-aminopyridine (4-AP, as well as 2-AP and 3-AP), 3,4- and 2,3-diaminopyridine, BaCl₂, CsCl, strychnine, phencyclidine, pyridostigmine, 9-aminoacridine, DuP-996 (3,3-bis (4-pyridinylmethyl)-1-phenylindolin-2-one; linopiridine), clofilium, quinidine, aminoquinolines and quinine.

Neurons (commercially available for culture (and for transfection and transformation), as well as postmitotic human CNS cells, for instance, from STRATAGENE, La Jolla, Calif.) are incubated in 12×75-mm polystyrene tubes with a radioligand (˜1-2×10³ Ci/mmol). Unless otherwise noted, cells are suspended in isotonic sucrose medium (Medium I) containing 10 mM NaHepes, 5 mM KCl, 5 mM NaCl, and 6 mM glucose, pH 8.4, and incubated with the radioligand for 1 h at room temperature on a rotary shaker. Nonspecific binding is determined in the presence of 10 nM native cold radioligand. Stock cell suspensions are diluted to give a final cell concentration of 2.5×10⁵ cells/ml in a total volume of 400 μl. At the end of the incubation period, samples are diluted with 4 ml of ice-cold Quench solution, which contain 200 mM NaCl, 20 mM Hepes (free acid), titrated to pH 8.0 with Trisbase. Quenched samples are filtered through GF/C glass microfiber filters, that had been presoaked in 0.6% polyethylenimine, and washed twice with ice-cold Quench solution. Triplicate samples are run for each experimental point. Standard deviation of the mean is typically less than 5%. Different cell preparations may produce somewhat different ratios of nonspecific/total radioligand binding. Stock solutions of the radioligand are prepared in 100 mM NaCl, 20 mM Tris-HCl, pH 7.4, 0.1% bovine serum albumin.

Analysis of Data—Data from saturation experiments are subjected to Scatchard analysis, and linear regression is performed to yield to equilibrium dissociation constant (K₄) and maximum receptor concentration (Bₘₐₓ). Correlation coefficients for these determinations are typically greater than 0.95. Data from competition experiments are analyzed by the standard method of Cheng and Prusoff to determine Kᵢ values. The dissociation rate constant (κᵢ) is determined directly from a first order plot of ligand dissociation versus time. The rate of ligand association (k₁) is determined from the equation k₁=k_{obe}([LR]ₑ/([L] [LR]ₘₐₓ)), where [L] is the concentration of ligand, [LR]ₑ is the concentration of the complex at equilibrium, [LR] ₘₐₓ is the maximum number of receptors present, and k_{obe} is the slope of the pseudo-first order plot ln ([LR]ₑ/{[L]ₑ−[LR]ₜ}] versus time. Association and dissociation rate of the radioligand are also determined by measuring the kinetics of radiolabeled entity binding at different ligand concentrations, determining k_{obe} at each concentration of ligand from semilogarithmic representations of these data, and determining k₁ and k-₁ from the slope and y intercept, respectively, of the plot of k_{obe} versus ligand concentration.

The Radioligand Binding Assays may be used with the present invention substanstially as described by Deutsch, C., et al., J. of Biological Chemistry, 266: No. 6, 3668-3674 (1991).

### EXAMPLE X

⁸⁶Rb Efflux Drug Screening Assay

Depolarization of human cells by high concentrations of extracellular potassium ions, leads to the activation of the voltage-gated potassium channels. The activity of such potassium channels is demonstrated to be effectively and rapidly monitored by tracking the efflux of ⁸⁶Rb from pre-loaded target cells in response to the depolarizing stimulus. The inclusion of compounds with unknown activity in the assay medium, results in the identification of modulators of potassium channels of which the subunit described herein, hKv9.3, is an integral member. See, Toral, J., et al., Use of Cultured Human Neuroblastoma Cells in Rapid Discovery of the Voltage-gated Potassium-channel Blockers, J. Pharm. Pharmacol., 46:731(1994). Blocking of individual K⁺ channels by a candidate compound results in a significant decrease in ⁸⁶Rb efflux which can be readily detected by this assay. Toral, J., et al. have successfully used this assay to discover a number of novel chemical structures capable of blocking the voltage-gated potassium channels in neurons and cardiocytes. The potassium-channel blocking activity of these compounds has been verified by electrophysiological techniques, as well as by ⁸⁶Rb efflux from cultured mammalian cells transfected with nucleic acids which encode potassium channel subunits.

The functional high-volume ⁸⁶Rb efflux assay is performed in 96-well microtitre plates, it represents a rapid and high-volume primary screening method for the detection and identification of potassium-channel modulators. The application is described for detecting modulators of potassium channels in cultured human neuroblastoma cells. Toral, J., et al., Use of Cultured Human Neuroblastoma Cells in Rapid Discovery of the Voltage-gated Potassium-channel Blockers, J. Pharm. Pharmacol., 46:731(1994).

Highly purified human NT2 neuron cells and hNT post mitotic cenral nervous system cells for differentiation toward phenotype, are available from STRATAGENE, La Jolla, Calif., for transfection to allow the study of potassium channel genes and assays described herein.

Buffers and Reagents

Buffer

MOPS-PSS, pH 7.4 (NaCl 120 mM; KCl 7.0 mM; CaCl₂ 2.0 MM; MgCl₂ 1.0 mM; ouabain 10 μM; 4-morpholinepropanesulphonic acid, MOPS 20 mM).

Depolarizing Solution

MOPS-PSS containing KCl (80 mM) replacing the equivalent concentrations of NaCl.

Candidate compounds are dissolved at a stock concentration of 10-100 mM, either in MOPS-PSS or dimethylsulphoxide (DMSO), and are subsequently diluted in the incubation buffer to the desired concentration. Candidate compounds are dissolved in MOPS-PSS containing bovine serum albumin (0.1 @ w/v) at 50-500 μM stock concentration.

Cell Culture and ⁸⁶Rb Loading

Human neuroblastoma cells TE671 are obtained from American Type Culture Collection (HTB 139) and are maintained at 37° C. in Dulbecco's Modified Eagle's Medium (DMEM) supplemented with 10% foetal bovine serum, 4.5 gL⁻¹ glucose and 2.0 mM L-glutamine. Cells are plated and loaded with ⁸⁶Rb in 96-well microtitre plates as described by Daniel, S., et al., J. Pharmacol. Methods, 25:185 (1991).

⁸⁶Rb Efflux Assay Procedure

The growth medium in the microtitre plate is discarded by a sharp flicking of the plate. The adherent cell layer is washed three times with 200 μL MOPS-PSS using a 12-channel pipetter. The cells are incubated for 30 min at room temperature, either with 200 μL MOPS-PSS, or 20 μL of the depolarizing solutions, in the presence or absence of a candidate compound potassium-channel blocker. Supernatant (150 μL) from each well is removed and counted. Cell layer is solubilized in 200 μL 0.1% Tween 20 in water and 150 μL is also counted in a Packard 2200 CA liquid scintillation counter. All supernatants are counted in 7.0 mL distilled water.

The percent efflux is calculated as follows:

$$\% total efflux = \frac{( counts {min}^{- 1} in supernatant )}{( counts {min}^{- 1} in supernatant + counts {min}^{- 1} in cell extraxct )} \times 100$$

and the value of percent net efflux is calculated as:

% net efflux=% total efflux−% basal efflux

where % total efflux is that induced by the depolarizing solution containing 100 mM KCl. The basal efflux is the efflux (leak) of ⁸⁶Rb observed in the physiological saline, MOPS-PSS.

### EXAMPLE XI

FLIPR™ Optical Screening System

Molecular Devices Corporation, Sunnyvale, Calif.

FLIPR (FLuorescent Imaging Plate Reader) was initially designed to perform as a large-volume screening tool for measurements of membrane potential of cells in 96 well microtiter plates. FLIPR is applied to fluorescent assays, such as in the measurement of particular intra-cellular ions and intracellular pH. FLIPR may also be used in non-fluorescent assays.

In fluorescent mode, FLIPR works by illuminating the bottom of a 96 well microplate and measuring the fluorescence from all 96 wells simultaneously using a cooled CCD camera. The excitation optics in FLIPR illuminate the plate from the bottom and the emission optics read the plate from the bottom. This requires that the microplate that is optically measured in the machine has a clear bottom.

The typical measurement assay consists of using one 96 well plate with cells and one (or two) 96 well plate with the candidate compounds to be screened.

The detection optics of FLIPR are based on cooled CCD (charge coupled device) technology. With each kinetic update, the system takes a picture of the bottom of a microplate, recording a signal for all the individual wells simultaneously. Enhanced sensitivity for cell-based assays is accomplished via optical detection, which allows for signal isolation on a cell monolayer. This technique is very important for assays where background fluorescence is present (such as from extra-cellular dye). In order to make FLIPR an effective screening tool, the instrument includes an accurate 96 well pipettor which can aspirate and dispense from two separate fluid addition microplates into a third microplate being optically tested. The entire system is controlled via a PC and Windows-based software interface. The interface provides flexible tools for assay development, quality control and data management.

Measurements are recorded for both voltage gated and ligand gated channels on many different cell lines including CHO, HEK, A10, ECV50, primarily cultured cells, and others. Visible wavelength indicators, namely Fluo-3 and Calcium Green-1 are redily used.

| | Suggested | | |
|---|---|---|---|
| Description | Supplier | Phone Number | Item Number |
| Black wall plates, clear | Corning/ | 800-492-1110 | 3603 |
| bottom, tissue culture | Costar | | |
| treated, sterile | | | |
| | Packard | 800-856-0734 | 6005182 |
| | Instrument | 203-639-2404 | |
| Nunc V-bottom 96 | Fisher | 800-766-7000 | 12-565-216 Nunc |
| well plate | | | part #249128 |
| Non-sterile lids for | PGC | 800-424-3300 | 5-6112-21 |
| Nunc plates | Scientific | | |
| Sterile basin for | Fisher | 800-766-7000 | 13-681-101 |
| multichannel pipettor | | | |
| Non-sterile basin for | Fisher | 800-766-7000 | 13-681-100 |
| multichannel pipettor | | | |
| Autotip, 96 tip rack, | Robins | 800-752-8585 | 1043-24-O |
| black tips, non-sterile, | Scientific | | |
| 200 μl | | | |
| Hank's Balanced Salt | Gibco | 800-828-6686 | 14065-056 |
| Solution | | | |
| HEPES buffer solution | Irvine | 800-437-5706 | 9319 |
| 1X | Scientific | | |
| Probenecid, crystalline | Sigma | 800-325-3010 | P8761 |
| Fluo-3 AM ester | Molecular | 800-438-2209 | F-1241 |
| | Probes | 541-465-8300 | |
| Ca-Green AM ester | Molecular | | C-3011 |
| | Probes | | |
| Pluronic acid | Molecular | | P-6867 |
| | Probes | | |
| DiBac(4)3, bis oxonol | Molecular | | B-438 |
| dye for membrane | Probes | | |
| potential | | | |
| BCECF AM ester for | Molecular | | B-1150 |
| intracellular pH | Probes | | |

### Components Needed for Dye Loading Cells:

250 mM Probenecid:

prepare fresh each day

710 mg Probenecid

5 ml 1.0N NaOH—solubilize in

5 ml Hanks'+20 mM Hepes

1 mM Dye:

Molecular Probes Fluo3, AM F-1241

Calcium Green 1, AM C-3011

1 mg Fluo3, AM or Ca-Greenl, AM

443 μl MDSO—solubilize dye

443 μl 20% Pluronic acid in DMSO

Store solution in aliquots at −20° C.

Can be frozen and thawed several times.

Alternatively, prepare a 2 mM dye solution that you keep frozen, and a 20% pluronic acid solution that you keep at room temperature, and mix the 2 solutions at a 1:1 ratio before use.

20% Pluronic acid:

Molecular Probes P-6867

Weigh in a tube and solubilize in DMSO at 37° C.,

for example: 400 mg/2 ml DMSO.

Let cool to room temperature and aliquote. Store at room temperature.

DMSO:

Use DMSO with low water content

For example; Sigma D2650 in 5 ml ampules

Other items needed and used in a cell culture facility:

12 channel pipettoro 50-200 μL, example Brinkman, part #5008130-3

Sterile pipette tips 200 μL, example E+K Scientific (tel: 408-378-2013), part #3507-R965) (10 racks of 96 tips)

Aspirator to remove medium from plate wells. Example: 12-pin manifold aspirator, part #851388, or 8-pin manifold aspirator, part #951381 (from Wheaton Science Products (tel: 800-225-1437)

5 mL, 10 mL, 25 mL sterile serological pipettes

Rechargeable pipettor for 2-25 mL pipettes

Sterile tissue culture water

Gloves

Culture medium to grow cells

EDTA and Trypsin/EDTA to life cells

Hemacytometer and counter

Sterile test tubes 15 mL and 50 mL

1 N NaOH solution

FLIPR Test

Membrane Potential

FLIPR was developed basically for the measurement of membrane potential using the voltage sensitive dye DiBAC(4)₃ (Molecular Probes B-438). The assay protocol was developed by Dr. Vince Groppi of Upjohn-Pharmacia in Kalamazoo, Mich.

Cell Culture

One criterion necessary for quality data from FLIPR is that the cells measured are at the bottom of the well. This does not necessary mean the cells are adherent. This necessity is due to the optical detection in FLIPR which enhances sensitivity to fluorescence from the bottom of the microplate well. Non-adherent cells can also be used, however not in suspension. Generally non-adherent cells are spun down, forming a pseudo-monolayer at the bottom of the plate.

Some cell types require a coating (e.g. Poly-D-Lysine) in order to insure adherence. Typical protocols would involve coating the microplates in a sterile environment before use.

The following procedure is used to prepare cells which overexpress the novel potassium channel subunit described herein, e.g. SEQ ID NO:2, for FLIPR

Day 1: Stock cultures of transformed or transfected heterologous expression cells are detached from T75 flasks with IX trypsin/EDTA. Typically 1 T75 flask will contain 2×10⁶ cells. The heterologous expression cells are diluted to approximately 0.25×10⁵ cells/ml and 0.2 ml of cells are distributed to each well of a 96 well plate. In this way each well is plated with 0.5×10⁴ A10 cells. In this manner 1 T75 flask will generate about four confluent 96 well plates after growing for 3 days.

Day 3: The heterologous expression cells are carefully fed with fresh medium. Care is taken not to disrupt the monolayer.

Day 4: Cells are ready for analysis in FLIPR.

Following this procedure, one should establish a uniform monolayer of cells in each well of a 96 well plate. If the monolayer appears non-uniform in any way, then the experiment is terminated and fresh cultures are prepared.

Having followed the above procedure to Day 4 (see previous section) it is now time to prepare the plates for testing in FLIPR. Based on their reluctance to overgrow, the A10 cells can probably be used successfully on Days 4-6.

Calibration

FLIPR can incorporate a calibration scheme using positive and negative control wells.

The negative controls are typically additions of a non-stimulating fluid buffer. The negative controls are used to make sure the instrument as well as the assay is working properly, yielding flat baselines and no response where there should not be any. The positive control wells are used to monitor changes in signal gain. The gain calibration is useful to compare all of the data within a single run to known positives, thereby calibrating the plate.

Preparation of Solutions (Membrane Potential Assay)

The following protocol is intended to provide enough solutions to test approximately 2 microplates:

1.) Prepare a 20 mM solution of HEPES Buffer in EBSS by adding 10 mL of 1M HEPES to 490 ml of EBSS. This solution is EBSS+H. Warm to 37 deg. C by incubation.

2.) Prepare 100 ml of EBSS+H containing 5 μM DiBAC, by adding 50 μM of 10 mM DiBAC to 100 ml of EBSS+H. This solution is EBSS+H+D. Keep at 37 deg. C. This solution will be used in the negative control wells, to wash the cells and also as the media in solution with the cells during testing. Approximately 60 ml will be used per testplate. The 10 mM DiBAC stock is made up from the powder in DMSO. For the DiBAC 10 mM stock: DiBAC normally comes in 25 mg quantities and it is useful to make around ˜20 300 μl aliquots in DMSO for freezing and later use.

3.) Prepare 50 ml of EBSS+H containing 10 μM DiBAC, by adding 50 μl of 10 mM DiBAC to 50 ml of EBSS+H. This solution is EBSS+H+D. Keep at 37 deg C. This 10 μM dye (2×) solution will be used in FLIPR to presoak the pipette tips during a run. DiBAC will absorb into plastic, and it is the stimulus compounds are prepared in DiBAC thereby maintaining the dye concentration in the optically measured microplate wells containing the cells.

4.) Prepare 100 ml of EBSS+H+D containing 300 mM KCl by adding 7.5 ml of 4M KCl to 92.5 ml of EBSS=H and 50 ml of 10 mM DiBAC. This solution is EBSS+H+D+300KCl. Keep at 37 deg C. This 10×KCl solution will be used for the positive control wells (20 ml addition to 180 ml: making the final concentration 30 mM KCl) in the addition plate.

Preparation of Cell Plate

1.) Remove growth media from cells with a multi-well pipettor. If using a 8 well pipettor do no more than 4 rows at a time to insure that the cells do not dry out. Add 250 μl of pre-warmed EBSS+H+D to the cells. Do this for the entire plate. This work should be carried out on a hot plate set to 37 deg C., to minimize temperature fluctuations. Do two washes with 250 μl of EBSS+H+D.

2.) Again doing 4 rows at a time, remove the 250 μl of EBSS+H+D and replace with 180 μl of fresh pre-warmed EBSS+H+D. Make sure that pipettor tips are pre-soaked and be consistent when handling dye washes to avoid variable dilution.

3.) After the cell plate is washed place it in the incubator to equilibrate to 37 deg C.

4.) The cells should be tested in FLIPR within 2 hours of the final wash.

Preparation of Candidate Compound (Drug) Addition Plate

1.) Place 220-250 μl of EBSS+H+D in the negative control wells. The pipettor will go down to the 50 μl fluid level and will typically only aspirate 20 μl per run, therefore this plate can be used several times. Clear microplates can be used for the stimulus plates. Using the FLIPR 96 well pipetter with a flat bottom plate will result in a dead volume of about 50 μl whereas a v-bottomed plate can have dead volumes as low as 10-20 μl.

2.) Place 220-250 μl of EBSS+H+D+300KCl in the designed positive control wells.

3.) If the unknowns are in a solvent, be sure to include the same concentration of solvent in the negative control wells. In our experience try to stay below 0.1% DMSO in possible.

4.) Place the drug addition plate in the incubator and equilibrate to 37 deg C.

Typical Measurement Sequence for Membrane Potential

Start-Up Procedure

1.) Turn on the laser water flow and electrical main disconnect.

2.) Turn on the laser. Typically, the laser will require about 15 to 30 minutes to stabilize. Coherent recommends a 30-minute warm-up stabilization period for the laser. They also recommend (due to tube lifetime) turning the laser off only if you plan on being down for more than 3 hours, otherwise leave the laser on.

3.) Turn on the computer, monitor and camera controller. Make sure that the CCD camera is at temperature before taking data with the camera. Proper temperature is indicated by a lit green a “status” light on the camera controller. Normally camera cool-down takes about 5 minutes.

4.) Power up the FLIPR enclosure. Make sure the PC is booted up before powering up the FLIPR enclosure. The system may lock up otherwise.

5.) Turn on the regulator for 80 psi air source.

6.) Clear any interlocks by making sure the cell drawer is in and the pipette tips are up.

7.) Start the airflow for the humidity chamber (temperature dependent assays only).

8.) Start the FLIPR code by double clicking on the FLIPR control software icon. The pipettor will also go through the normal homing cycle on start-up. If temperature control is desired, and it not set up in the setup.flp file as default on, turn it on via the Setup “heaters” pull down option. It will take approximately 30 minutes for the temperature sensors to stabilize. Recall that the temperature control option requires the low-pressure regulated air source be turned on also.

9.) Wait for the temperature warning indicators to clear on the display window if temperature control is being used.

Data Collection

Because of the strong temperature dependence of DiBAC, it is a good idea to immediately place the cells in the FLIPR cell incubation chamber after removing them from the incubator. Once the cells are place in solution with DiBAC, they should be tested within 2 hours.

To reduce the temperature stabilization time, the cell plate and the addition plate should be removed from the incubator and put into the cell incubation chamber in FLIPR as quickly as possible. The more careful the operator is in handling the plates, the quicker the DiBAC temperature stabilizes and the quicker the fluorescent baselines stabilizes.

The operator then needs to set up the parameters to be used in the experiment. Choosing the Experiment option under the Setup pull-down menu does this.

Typical System Setup for Measurements of Membrane Potential

System gain parameters:

Assuming the laser is set to around 300 mW

Since DiBAC is bright, the F/stop should be set around F5.6, i.e. stopped down a little Camera Exposure time around 0.4 seconds (how to set this is described below).

General Setup Parameters:

a.) Exposure Length: (camera integration time): usually around 0.4 seconds

b.) Filter #1 (standard filter)

c.) Presoak tips (usually checked, usually from left tray)

d.) Multiple additions

e.) Automation: user's choice of options

Under the First Sequence Definition

a.) Sample Interval (typically 20 seconds)

b.) Sample count (depends on length of experiment desired), usually 15 minutes is enough for DiBAC, therefore a 20 second updates, and 15 minute total run time, 45 samples would be taken.

c.) Second interval (usually not necessary with DiBAC, generally only used for fast updates)

d.) Fluid addition checked active

e.) Fluid volume to add (usually 20 μl, as DiBAC is temperature sensitive and smaller addition volumes are preferred.

f.) Dispense speed (depends on cell type, 20 μl/sec is slow, 80 μl is fast, better mixing the faster the mix speed, depends on what the cells will withstand without being relocated on the bottom of the plate.

Under Pipetting

a.) Mix volume (generally 40 μl)

b.) Number of mixes (generally 3, do mixing because of adding a small volume to a large volume, as well as the kinetics of DiBAC are slow enough to allow for omitting between updates.

c.) Tip position, uncheck, i.e. tips are out of well during data collection, since DiBAC experiment is slow, the pipettor has time to go up and down into the fluid between sample points, by unchecking this box one can insure that compound does not leach out into the cell plate prior to the pre-programmed delivery.

d.) Fluid Addition, generally for DiBAC this will be checked, that is well will remove the same amount of fluid which was added, in this example 20 μl. This insures that there is no fluid height dependence associated with adding a very fluorescent background component, that being the DiBAC in solution with the addition compounds. This may or may not be necessary. With this box unchecked, the fluid will be added, changing the total well volume to 200 μl (assuming 20 μl to 180 μl already added in the cell plate).

Once the experiment has been defined, the user will perform a signal level check using the “light bulb” or Run “Signal Test” option. This takes a camera exposure suing the current exposure time defined in the Setup Experiment window and displays the signal counts for all 96 wells of the plate. It also displays some statistics of the average, minimum and maximum signal counts for the exposure. Since the total dynamic range of the camera is 65,000 counts is it a good idea to work with signal counts in the range of 25,000 to 30,000 DiBAC. At these gain levels, a good physiological DiBAC signal (e.g. a membrane depolarization) will be on the order of 5,000 counts. Thus starting with a basal fluorescent range of 25 to 30,000 counts helps to insure that the individual camera pixels will not saturate during the data collection.

If the signal counts are too high or low, shortening or lengthening respectively, the camera exposure interval is recommended until the correct signal level is achieved. If exposure times less than 0.4 seconds are required to come down to the desired video levels, then turn down the laser source power or alternatively, stop down the camera aperture. Each F/stop increase of the camera cuts the detection sensitivity by a factor of two. General ranges for the system gain parameters are as follows:

Laser power: run between 150 mW and 1 Watt, it is advised to leave around 300 mW so laser warms up at a consistent place every day, increase power when more gain is needed.

Camera F/stop (run between F/2 and F/22), note that each stop is a factor of 2 in sensitivity with F/2 being the largest aperture, with the most sensitivity. This is the easiest thing to change to et rid of light, for example when running the DiBAC assay.

Camera Exposure time: Run above 0.2 seconds, for kinetic (multiple frame) data. This is because at very short exposure times the mechanical jitter in the opening and closing of the camera shutter can add noise to the temporal fluorescent traces. This is not an issue for a single (non-kinetic) exposure.

After the signal level is set, the operator is ready to start a baseline stability check. This is usually a good idea to insure that the fluorescent baselines are under thermal equilibrium resulting in flat baseline fluorescence. This can be done by starting an experiment, usually with 20 second time updates, with the pipetting deactivated. This is more of an issue with DiBAC assays than with intracellular calcium assays. Depending on the temperature of the fluid when the cell plate with placed in FLIPR, stabilization can take a few minutes or up to 20 minutes.

Assuming the baselines are flat, then it is time to start the assay. Hitting the stop icon at any time can stop the baseline. Once the user is satisfied that the baselines are flat enough, the pipettor should be re-activated in the Experimental Setup window; “First sequence” dialog page.

The experiment will continue, with the real-time display updating the measured fluorescent data values, until the total number of measurements has been completed. At this point the pipettor will return to the pipette load station on the right side of FLIPR.

Once the run is completed the user is then ready to export (or process) the data. This basically converts the raw data files, which have already been compressed and stored on the hard disk, into fluorescent signal counts per well. Exporting of data is covered in detail in Section 3.5.

Notes

After dye loading, several washes in a non-fluorescent salt buffer will be required to reduce signal artifacts associated with the presence of extra-cellular dye as well as background associated with fluorescent entities in media. The main artifact present with improper washing is a sharp signal decline upon addition of the stimulus due to the dilution of the strong background component. Recall that generally the compounds are mixed up in a non-fluorescent salt solution, e.g. Hanks, Earles etc. and that large volume additions are made (e.g. 50 μl to 100 μI) to insure rapid mixing.

The cell wash buffer should be the same solution used with the cells during testing. Likewise, this same buffer should be used to prepare the stimulus compounds to avoid unwanted pH, osmolarity, or morphology changes in the cells during a kinetic experiment. Typically balanced salt solutions like HBS, PBS or EBSS will be used. It is very important that the compounds are prepared in “exactly” the same buffer as the cells are washed and sitting in prior to running the experiment.

A typical protocol would be to load in dye for 1.5 hours, wash 3.4 times with buffer (200 μl per well), for example using a gentle washer such as the Denley CellWash. Exactly how many washes are necessary will be cell type dependent. Sometimes a wait period between washes (two before, two after) is good to allow the cells to re-equilibrate to a lower extra-cellular dye concentration, before doing the final wash. If the cells are only slightly adherent, there will be a trade-off between washing the cells thoroughly to remove extra-cellular dye, and washing cells off the plates.

The pipettor dispense speed should generally be about 50 μl/second. However, if the cells are only slightly adherent, the user may need to reduce the dispense speed. A slow dispense speed is on the order of 20 μl/second, and a fast dispense speed is around 80 μl/sec. These values must be experimentally determined for each cell type, but generally it is preferable to dispense as fast as possible to enhance mixing. The trade-off is that the pipetting speed cannot be so forceful so as to dislodge the cells at the bottom of the well. This will in effect removed the fluorescent cells from the field of view of the camera pixels, thereby causing large decreases (artifacts) in the fluorescent traces upon the fluid addition.

At the beginning of a data run, it is useful to perform a “signal test” to check the dye loading level in the cells. When establishing loading level protocols, it is useful not to load dye into the entire microplate to get a reading on the background fluorescent counts. These unloaded wells should be treated just like the loaded wells in terms of wash steps etc. The dominant background component (assuming a salt solution is used to the buffer during the experiment) will be fluorescence from the plastic microplates. Most salt buffer solutions have negligible fluorescence at excitation wavelengths of 488 nm.

Work with starting fluorescent levels (above background) of about 10,000 to 20,000 counts (recall saturation is 65,000 counts) is recommended for basal ion levels. Again, depending on loading levels, this should be achievable with approximately 0.300 W of laser power, 0.4 sec of integration time and an F/stop setting of F/2.

Specific Dye Loading Protocol for Heterologous Overexpressor Cells

1.) Plate cells to confluence: need to be adherent

2.) Make ˜2 mM dye stock in anhydrous DMSO

3.) Make 1 mM dye stock by diluting with 20% w/v pluronic acid (in our experience very necessary)

4.) Make 4 μM dye loading stock by adding 40 μl of 1 mM stock to 10 mls of loading buffer or media. This will do about 1 plate.

5.) Remove growth media and serum from plated cells. Alternatively, if loading in media and serum, just add dye/media/serum to existing media/serum. This removes an aspirate step, however dye concentration if reduced.

6.) Load cells with 4 μM dye (˜100 ul per well) for 1.0 to 1.5 hours)

7.) Wash cells with buffer solution twice. You want a good wash so use volumes of at least 200 Ξl per well.

8.) Re-incubate cells for 15 minutes.

9.) Wash twice more with buffer, leaving 100-150 μl in wells. The actual volume left depends on the amount of dilution desired in the preparation of the stimulus plate. It is important that the final volumes be consistent so as to produce good well to well consistency. It may be appropriate to do 4 buffer washes consecutively, without reincubation. This is usually empirically determined based on whether or not a dye leakage artifact is present in the negative control wells.

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

What is claimed is:

1. A purified polynucleotide comprising a nucleic acid sequence encoding the polypeptide having the sequence substantially as depicted in SEQ ID NO:3 or a pharmacologically active fragment thereof.

2. The polynucleotide of claim 1 wherein the polynucleotide sequence comprises the sequence substantially as depicted in SEQ ID NO:2.

3. An expression vector comprising the polynucleotide of claim 1.

4. An antisense molecule comprising the complement of the polynucleotide of claim 2 or a biologically-effective portion thereof.

5. A host cell transformed with the expression vector of claim 3.

6. A purified polypeptide comprising the amino acid sequence substantially as depicted in SEQ ID NO:3.

7. An antibody specific for the polypeptide of claim 6.

8. A method for producing cells which express a polypeptide substantially as depicted in SEQ ID NO:3, said method comprising
a) culturing a host cell according to claim 5 under conditions suitable for the expression of said polypeptide.

9. A method for producing a polypeptide having the amino acid sequence substantially as depicted in SEQ ID NO:3, said method comprising the steps of:
a) culturing a host cell according to claim 5 under conditions suitable for the expression of said polypeptide, and
b) recovering said polypeptide from the host cell culture.

10. A diagnostic composition for the identification of a polypeptide substantially as depicted in SEQ ID NO:3 comprising the antibody of claim 7.

11. A method of screening a plurality of compounds for binding affinity with the human voltage-gated potassium channel subunit of claim 6, said method comprising the steps of:
a) providing a plurality of compounds;
b) combining the compounds with the human voltage-gated potassium channel subunit for a time sufficient for the compound to bind the subunit; and
c) detecting and recovering the compound which binds the human voltage-gated potassium channel subunit.

12. A method of identifying compounds that modulate the pharmacological activity of hKv9.3, comprising:
a) combining a candidate compound modulator of pharmacological activity with a potassium channel subunit polypeptide comprising the sequence substantially as depicted in SEQ ID NO:3, and
b) measuring an effect of the candidate compound modulator on the pharmacological activity of hKv9.3.

13. A method of identifying compounds that modulate the pharmacological activity of hKv9.3, comprising:
a) providing a first host cell which expresses at least one potassium channel subunit to produce a first potassium channel; and
b) providing a second host cell which co-expresses a potassium channel polypeptide comprising the sequence substantially as depicted in SEQ ID NO:3 with the said at least one potassium channel subunit to produce a second potassium channel; and
c) comparing the biological activity of the first potassium channel to the biological activity of the second potassium channel and identifying the pharmacological activity contributed by hKv9.3 to the potassium channel; and
d) combining a candidate compound modulator of hKv9.3 pharmacological activity with said second host cell; and
e) measuring an effect of the candidate compound modulator on the pharmacological activity contributed by hKv9.3 to the second potassium channel.

14. A method of identifying compounds that modulate the biological activity of a potassium channel of which hKv9.3 is an integral member, comprising:
a) providing a host cell which co-expresses at least one potassium channel subunit and a subunit comprised of the sequence substantially as depicted in SEQ ID NO:3 to produce a potassium channel; and
b) measuring the biological activity of the channel; and
c) combining a candidate compound modulator with said host cell; and
d) measuring the effect of the candidate compound modulator on the biological activity of the potassium channel.

15. A method according to claim 14 of identifying compounds that modulate the biological activity of a potassium channel of which hKv9.3 is an integral member, comprising:
a) providing a host cell which co-expresses at least one potassium channel subunit selected from the group consisting essentially of (Kv1.1, Kv1.2, Kv1.3, Kv1.4, Kv1.5, Kv1.6, Kv1.7, Kv2.1, Kv2.2, Kv2.3, Kv3.1, Kv3.2, Kv3.3, Kv3.4, Kv4.1, Kv4.2, Kv4.3, Kv5.1, Kv6.1, Kv7.1, Kv8.1, Kv9.1, Kv9.2, Kv9.3, KQT1, KQT2, KQT3, KCNQ2, KCNQ3, ISK, HERG1, HERG2, ELK1, ELK2) and a subunit comprised of the sequence substantially as depicted in SEQ ID NO:3 to produce a potassium channel; and
b) measuring the biological activity of the channel; and
c) combining a candidate compound modulator with said host cell; and
d) measuring the effect of the candidate compound modulator on the biological activity of the potassium channel.

16. A method of treatment of a patient in need of such treatment for a condition which is mediated by the biological activity of a human potassium channel, comprising administration of hKv9.3 substantially as depicted in SEQ ID NO:3 or a pharmacologically active fragment thereof.

17. A method of treatment of a patient in need of such treatment for a condition which is mediated by the biological activity of a human potassium channel, comprising administration of a nucleic acid substantially as depicted in SEQ ID NO:1 or a biologically-effective fragment thereof.

18. A method of treatment of a patient in need of such treatment for a condition which is mediated by the biological activity of a human potassium channel, comprising administration of a antisense molecule according to claim 4 or a biologically-effective fragment thereof.

19. A compound that modulates the pharmacological activity of hKv9.3 identified by the method of claim 12.

20. A compound that modulates the pharmacological activity of hKv9.3 identified by the method of claim 13.

21. A compound that modulates the the biological activity of a potassium channel of which hKv9.3 is an integral member identified by the method of claim 14.

22. A pharmaceutical composition comprising a compound that modulates the pharmacological activity of hKv9.3 according to claim 20.

23. A pharmaceutical composition comprising a compound that modulates the biological activity of a potassium channel of which hKv9.3 is an integral member according to claim 21.

24. A method of treatment of a patient in need of such treatment for a condition which is mediated by the biological activity of a human potassium channel, comprising administration of a modulating compound according to claim 20.

25. A method of treatment of a patient in need of such treatment for a condition which is mediated by the biological activity of a human potassium channel, comprising administration of a modulating compound according to claim 21.


SEQUENCE LISTING (11 sequences)

SEQ ID NO: 1 - DNA (cDNA), 2421 bp; single-stranded, unknown
cgcccgggcaggtccagcgatgccttctggaccacactctgtcctcgccgaggtcctggtttgttcctcaacgtcggctgaaggcatcgtcccctgacttcctcttccattcgtccttccgaaaggggacccgactggttcctcttctacatatgaggcgcatctgcttggtgcaaatgtccaagcacctcttctgactcggtgagagtgattttccagtgattgctttggcctgtacaaccagagaacaggattcttcccttctttttggccaccaaatgcctatgtgcaccacacattccagtgtgctgagaagggcagagcttcttggatgatgatggacgtcccaccgggcaggatgaaggcagagcgtgtggcatctccacctcaagggtgcagcctgatcttcctcttctcccttgccagccagcactctgccttctgtatccaccatggtgtttggtgagtttttccatcgccctggacaagacgaggaacttgtcaacctgaatgtggggggctttaagcagtctgttgaccaaagcaccctcctgcggtttcctcacaccagactggggaagctgcttacttgccattctgaagaggccattctggagctgtgtgatgattacagtgtggccgataaggaatactactttgatcggaatccctccttgttcagatatgttttgaatttttattacacggggaagctgcatgtcatggaggagctgtgcgtattctcattctgccaggagatcgagtactggggcatcaacgagctcttcattgattcttgctgcagcaatcgctaccaggaacgcaaggaggaaaaccacgagaaggactgggaccagaaaagccatgatgtgagtaccgactcctcgtttgaagagtcgtctctgtttgagaaagagctggagaagtttgacacactgcgatttggtcagctccggaagaaaatctggattagaatggagaatccagcgtactgcctgtccgctaagcttatcgctatctcctccttgagcgtggtgctggcctccatcgtggccatgtgcgttcacagcatgtcggagttccagaatgaggatggagaagtggatgatccggtgctggaaggagtggagatcgcgtgcattgcctggttcaccggggagcttgccgtccggctggctgccgctccttgtcaaaagaaattctggaaaaaccctctgaacatcattgactttgtctctattattcccttctatgccacgttggctgtagacaccaaggaggaagagagtgaggatattgagaacatgggcaaggtggtccagatcctacggcttatgaggattttccgaattctaaagcttgcccggcactcggtaggacttcggtctctaggtgccacactgagacacagctaccatgaagttgggcttctgcttctcttcctctctgtgggcatttccattttctctgtgcttatctactccgtggagaaagatgaccacacatccagcctcaccagcatccccatctgctggtggtgggccaccatcagcatgacaactgtgggctatggagacacccacccggtcaccttggcgggaaagctcatcgccagcacatgcatcatctgtggcatcttggtggtggcccttcccatcaccatcatcttcaacaagttttccaagtactaccagaagcaaaaggacattgatgtggaccagtgcagtgaggatgcaccagagaagtgtcatgagctaccttactttaacattagggatatatatgcacagcggatgcacaccttcattaccagtctctcttctgtaggcattgtggtgagcgatcctgactccacagatgcttcaagcattgaagacaatgaggacatttgtaacaccacctccttggagaattgcacagcaaaatgagcgggggtgtttgtgcctgtttctcttatcctttcccgacattaggttaacacagctttataaacctcagtgggttcgttaaaatcatttaattctcagggtgtacctttcagccatagttggacattcattgctgaattctgaaatgatagaattgtctttatttttctctgtgaggtcaattaaatgccttgttctgaaatttattttttacaagagagagttgtgatatagtttggaatataagataaatggtattgggtggggtttgtggctacagcttatgcatcattctgtgtttgtcatttactcacattgagctaactttaaattactgacaagtagaatcaaaggtgcagctgactgagacgacatgcatgtaagatccacaaaatgagacaatgcatgtaaatccatgctcatgttctaaacatggaaactaggagcctaataaacttcctaattcagtatggaaaaaaaaaaaaaaaaaaaa

SEQ ID NO: 2 - DNA (cDNA), 1476 bp; single-stranded, unknown
atggtgtttggtgagtttttccatcgccctggacaagacgaggaacttgtcaacctgaatgtggggggctttaagcagtctgttgaccaaagcaccctcctgcggtttcctcacaccagactggggaagctgcttacttgccattctgaagaggccattctggagctgtgtgatgattacagtgtggccgataaggaatactactttgatcggaatccctccttgttcagatatgttttgaatttttattacacggggaagctgcatgtcatggaggagctgtgcgtattctcattctgccaggagatcgagtactggggcatcaacgagctcttcattgattcttgctgcagcaatcgctaccaggaacgcaaggaggaaaaccacgagaaggactgggaccagaaaagccatgatgtgagtaccgactcctcgtttgaagagtcgtctctgtttgagaaagagctggagaagtttgacacactgcgatttggtcagctccggaagaaaatctggattagaatggagaatccagcgtactgcctgtccgctaagcttatcgctatctcctccttgagcgtggtgctggcctccatcgtggccatgtgcgttcacagcatgtcggagttccagaatgaggatggagaagtggatgatccggtgctggaaggagtggagatcgcgtgcattgcctggttcaccggggagcttgccgtccggctggctgccgctccttgtcaaaagaaattctggaaaaaccctctgaacatcattgactttgtctctattattcccttctatgccacgttggctgtagacaccaaggaggaagagagtgaggatattgagaacatgggcaaggtggtccagatcctacggcttatgaggattttccgaattctaaagcttgcccggcactcggtaggacttcggtctctaggtgccacactgagacacagctaccatgaagttgggcttctgcttctcttcctctctgtgggcatttccattttctctgtgcttatctactccgtggagaaagatgaccacacatccagcctcaccagcatccccatctgctggtggtgggccaccatcagcatgacaactgtgggctatggagacacccacccggtcaccttggcgggaaagctcatcgccagcacatgcatcatctgtggcatcttggtggtggcccttcccatcaccatcatcttcaacaagttttccaagtactaccagaagcaaaaggacattgatgtggaccagtgcagtgaggatgcaccagagaagtgtcatgagctaccttactttaacattagggatatatatgcacagcggatgcacaccttcattaccagtctctcttctgtaggcattgtggtgagcgatcctgactccacagatgcttcaagcattgaagacaatgaggacatttgtaacaccacctccttggagaattgcacagcaaaatga

SEQ ID NO: 3 - protein, 491 aa
MVFGEFFHRPGQDEELVNLNVGGFKQSVDQSTLLRFPHTRLGKLLTCHSEEAILELCDDYSVADKEYYFDRNPSLFRYVLNFYYTGKLHVMEELCVFSFCQEIEYWGINELFIDSCCSNRYQERKEENHEKDWDQKSHDVSTDSSFEESSLFEKELEKFDTLRFGQLRKKIWIRMENPAYCLSAKLIAISSLSVVLASIVAMCVHSMSEFQNEDGEVDDPVLEGVEIACIAWFTGELAVRLAAAPCQKKFWKNPLNIIDFVSIIPFYATLAVDTKEEESEDIENMGKVVQILRLMRIFRILKLARHSVGLRSLGATLRHSYHEVGLLLLFLSVGISIFSVLIYSVEKDDHTSSLTSIPICWWWATISMTTVGYGDTHPVTLAGKLIASTCIICGILVVALPITIIFNKFSKYYQKQKDIDVDQCSEDAPEKCHELPYFNIRDIYAQRMHTFITSLSSVGIVVSDPDSTDASSIEDNEDICNTTSLENCTAK

SEQ ID NO: 4 - protein, 491 aa
MVFGEFFHRPGQDEELVNLNVGGFKQSVDQSTLLRFPHTRLGKLLTCHSEEAILELCDDYSVADKEYYFDRNPSLFRYVLNFYYTGKLHVMEELCVFSFCQEIEYWGINELFFDSCCSSRYQERKEESHEKDWDQKSNDVSTDSSFEESSLFEKELEKFDELRFGQLRKKIWIRMENPAYCLSAKLIAISSLSVVLASIVAMCVHSMSEFQNEDGEVDDPVLEGVEIACIAWFTGELAIRLVAAPSQKKFWKNPLNIIDFVSIIPFYATLAVDTKEEESEDIENMGKVVQILRLMRIFRILKLARHSVGLRSLGATLRHSYHEVGLLLLFLSVGISIFSVLIYSVEKDELASSLTSIPICWWWATISMTTVGYGDTHPVTLAGKIIASTCIICGILVVALPITIIFNKFSKYYQKQKDMDVDQCSEDPPEKCHELPYFNIRDVYAQQVHAFITSLSSIGIVVSDPDSTDASSVEDNEDAYNTASLENCTAK

SEQ ID NO: 5 - protein, 20 aa
PDAFWWAVVSMTTVGYGDMY

SEQ ID NO: 6 - DNA (Other), 52 bp; single-stranded, linear
tcagtgtggcacctagagaccgaagttcagtgtggcacctagagaccgaagt

SEQ ID NO: 7 - DNA (Other), 27 bp; single-stranded, linear
tcctcactctcttcctccttggtgtct

SEQ ID NO: 8 - DNA (Other), 39 bp; single-stranded, linear
ccgctcgagtcattttgctgtgcaattctccaaggaggt

SEQ ID NO: 9 - DNA (Other), 39 bp; single-stranded, linear
tccccgcggtccaccatggtgtttggtgagtttttccat

SEQ ID NO: 10 - DNA (cDNA), 2565 bp; unknown-stranded, unknown
atgacgaagcatggctcccgctccaccagctcgctgccgcccgagcccatggagatcgtgcgcagcaaggcgtgctctcggcgggtccgcctcaacgtcggggggctggcgcacgaggtactctggcgtaccctggaccgcctgccccgcacgcggctgggcaagctccgcgactgcaacacgcacgactcgctgctcgaggtgtgcgatgactacagcctcgacgacaacgagtacttctttgaccgccacccgggcgccttcacctccatcctcaacttctaccgcactgggcgactgcacatgatggaggagatgtgcgcgctcagcttcagccaagagctcgactactggggcatcgacgagatctacctggagtcctgctgccaggcccgctaccaccagaagaaagagcagatgaacgaggagctcaagcgtgaggccgagactctacgggagcgggaaggcgaggagttcgataacacgtgctgcgcagagaagaggaaaaaactctgggacctactggagaagcccaattcctctgtggctgccaagatccttgccataatttccatcatgttcatcgtcctctccaccattgccctgtccctcaacacgctgcctgagctacagagcctcgatgagttcggccagtccacagacaacccccagctggcccacgtggaggccgtgtgcatcgcatggttcaccatggagtacctgctgaggttcctctcctcgcccaagaagtggaagttcttcaagggcccactcaatgccattgacttgttggccattctgccatactatgtcaccattttcctcaccgaatccaacaagagcgtgctgcaattccagaatgtccgccgcgtggtccagatcttccgcatcatgcgaattctccgcatccttaagcttgcacgccactccactggcctccagtctctgggcttcactttgcggaggagctacaatgagttgggcttgctcatcctcttccttgccatgggcattatgatcttctccagccttgtcttctttgctgagaaggatgaggacgacaccaagttcaaaagcatcccagcctctttctggtgggccaccatcaccatgactactgttgggtatggagacatctaccccaagactctcctggggaaaattgttgggggactctgctgcattgcaggagtcctggtgattgctcttcccatccccatcatcgtcaataacttctctgagttctataaggagcagaagagacaggagaaagcaatcaaacggcgagaggctctggagagagccaagaggaatggcagcatcgtatccatgaacatgaaggatgcttttgcccggagcattgagatgatggacattgtggttgagaaaaatggggagaatatgggtaagaaagacaaagtacaagataaccacttgtctcctaacaaatggaaatggacaaagaggacactgtctgaaaccagctcaagtaagtcctttgaaaccaaggaacagggatcccctgaaaaagccagatcgtcttctagtcctcagcacctgaacgttcagcagttggaagacatgtacaataagatggccaagacccaatcccaacccatcctcaataccaaggagtcagcagcacagagcaaaccaaaggaagaacttgaaatggagagtatccccagccccgtagcccctctgcccactcgcacagaaggggtcattgacatgcgaagtatgtcaagcattgatagtttcattagctgtgccacagacttccctgaggccaccagattctcccacagccctttgacatcactccccagcaagactgggggcagcacagccccagaagtgggctggcggggagctctgggtgccagtggtggtaggtttgtggaggccaaccccagccctgatgccagccagcactctagtttcttcatcgagagccccaagagttccatgaaaactaacaaccctttgaagctccgagcacttaaagtcaacttcatggagggtgaccccagtccactcctccccgttctagggatgtaccatgaccctctcaggaaccgggggagtgctgcggctgctgtcgctggactggagtgtgccacgcttttggacaaggctgtgctgagcccagagtcctccatctacaccacagcaagtgctaagacacccccccggtctcctgagaaacacacagcaatagcgttcaactttgaggcgggtgtccaccagtacattgacgcagacacagatgatgagggacagctgctctacagtgtggactccagcccccccaaaagcctccctgggagcaccagtccgaagttcagcacggggacaagatcggagaaaaaccactttgaaagctcccctttacccacctcccctaagttcttaaggcagaactgtatttactccacagaagcattgactggaaaaggccccagtggtcaggaaaagtgcaaacttgagaaccacatctcccctgacgtccgtgtgttgccagggggaggagcccatggaagcacacgagatcagagcatctga

SEQ ID NO: 11 - protein, 13 aa
RIMRILRILKLAR